(19) United States
(12) Patent Application Publication
(10) Pub. No.: US 2026/0253114 A1
(43) Pub. Date: Aug. 27, 2026

(54) SYSTEM AND METHOD FOR DYNAMIC INVOICE GENERATION AND APPROVAL MANAGEMENT

(71) Applicant: West Affum Holdings DAC, Dublin (IE)

(72) Inventors: Justin P. Beurmann, Lake Orion, MI (US); Maruthi S. Karpey, McKinney, TX (US); Christina M. Boster, Renton, WA (US); Jennifer D. Imerini, St. Augustine, FL (US); Shan M. Wilson, Poulsbo, WA (US); Karen D. Colbert, Kirkland, WA (US); Leah Jiang, Kirkland, WA (US); Ronald G. Lordi, Hot Springs Villa, AR (US); Sarah Q. Minici, Yorba Linda, CA (US); Jennifer L. Adams, Ballwin, MO (US); Himanshu Shekar, Noida (IN); Jesse G. Nord, Everett, WA (US); Robert A. Koll, Seattle, WA (US)

(73) Assignee: West Affum Holdings DAC, Dublin (IE)

(21) Appl. No.: 19/548,626

(22) Filed: Feb. 24, 2026

Related U.S. Application Data
(60) Provisional application No. 63/763,133, filed on Feb. 25, 2025.

(51) Int. Cl. G06Q 30/04; G06Q 30/0283; G16H 40/40

(52) U.S. Cl. CPC G06Q 30/04; G06Q 30/0283; G16H 40/40

(57) ABSTRACT

A system for invoice management related to wearable medical devices is disclosed. The system includes a processor and a memory including instructions, which when executed, cause the processor to receive a user creation input to create an invoice for tasks associated with a wearable medical device. The processor, additionally, displays invoice entry options based on the invoice type. The invoice entry options are associated with the tasks and/or expenses. Further, the processor receives selected invoice entries of the invoice entry option and determines, via a fee schedule, a fee for each of the one or more selected invoice entries. Furthermore, the processor generates the invoice including the selected invoice entries and the fee associated with the selected invoice entries.

### CROSS-REFERENCE TO RELATED APPLICATIONS

This application claims priority to and the benefit of the provisional patent application No. 63/763,133 titled "INVOICE MANAGEMENT SYSTEM IN AN ENTERPRISE AND METHOD THEREOF," filed in the United States Patent and Trademark Office on February 25, 2025. The specification of the above referenced patent application is incorporated herein by reference in its entirety.

### TECHNICAL FIELD

The present technology relates to the field of invoice management systems, and more particularly, but not by way of limitation, the present technology relates to a method for managing, submitting, and approving invoices within an enterprise using a cloud-based platform.

### BACKGROUND

In enterprises that rely on distributed workforces or contract-based personnel, managing invoices for services and expenses can be an intricate and time-consuming process. Contractors or field representatives often prepare invoices manually using general-purpose applications such as spreadsheets or word processors and submit them through email or other informal channels. These manual processes introduce inefficiencies, such as inconsistent invoice formats, errors in data entry, missing or incomplete information, and delays in payment processing. Finance departments, in turn, face ongoing challenges in maintaining accuracy, reconciling invoices with actual work performed, and tracking submission or approval status across multiple service engagements.

Various digital invoicing and enterprise management platforms have been developed that enable users to generate and submit invoices electronically. Such systems typically allow for digital approval routing, basic automation of invoice creation, and centralized storage of invoice records. These tools have helped reduce paper-based inefficiencies and provide better visibility into invoice processing compared to traditional manual methods.

However, existing invoicing and approval systems still present difficulties in accommodating diverse billing structures, varied service types, and evolving departmental policies. Many of these systems remain limited in flexibility and require manual configuration or maintenance to adapt to organizational changes. As a result, enterprises continue to experience inconsistencies in data entry, redundant manual actions, delays in approvals, and limited transparency in financial tracking and reporting. Accordingly, there remains a need for improved systems and methods that can address these shortcomings and enhance overall efficiency in enterprise invoice management.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

According to an embodiment of the present disclosure, a method for generating aninvoice related to servicing a wearable medical device for a patient is provided. The method includes receiving, via an invoice creation interface of an invoice management system, a user creation input to create the invoice for one or more tasks associated with the wearable medical device, the user creation input comprising an invoice type. In addition, the method includes dynamically displaying, via the invoice creation interface, a plurality of invoice entry options based on the invoice type, where the plurality of invoice entry options are associated with the one or more tasks and/or one or more expenses. Further, the method includes receiving, via the invoice creation interface, one or more selected invoice entries of the plurality of invoice entry options. Furthermore, the method includes determining, via a fee schedule of the invoice management system, a fee for each of the one or more selected invoice entries. Also, the method includes generating the invoice comprising the one or more selected invoice entries and the fee associated with the one or more selected invoice entries.

A further embodiment of the present disclosure includes a computer system comprising at least one processor and a memory operatively associated with the at least one processor, the memory including instructions stored thereon that, when executed by the at least one processor, cause the at least one processor to perform the above-mentioned operations.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

Example embodiments will be described and explained with additional specificity anddetail through the use of the accompanying drawings in which:

FIG. 1 illustrates a computer environment for generating and managing invoicesrelated to servicing a wearable medical device for a patient, in accordance with an embodiment of the present disclosure;

FIG. 2 illustrates a data model depicting the interaction between various data components and entities during invoice generation, processing, and approval within the computer environment, in accordance with an embodiment of the present disclosure;

FIG. 3 illustrates an interface representation depicting a graphical layout of a newinvoice within invoice creation interface of the invoice management system, in accordance with an embodiment of the present disclosure;

FIG. 4 illustrates an interface representation depicting creation of a new invoice line entry within invoice creation interface of the invoice management system, in accordance with an embodiment of the present disclosure; and

FIG. 5 illustrates a workflow diagram representing routing and approval of different invoice types and their corresponding invoice entries within the invoice management system, in accordance with an embodiment of the present disclosure.

FIG. 6 illustrates a screen flow representing an operational sequence for invoicecreation and data entry within the invoice management system, in accordance with an embodiment of the present disclosure.

FIGS. 7A and 7B illustrate an interface representation depicting a process for uploading supporting documents corresponding to an invoice line entry within the invoice management system, in accordance with an embodiment of the present disclosure.

FIG. 8 illustrates an interface representation depicting an invoice listing andmanagement view within the invoice management system, in accordance with an embodiment of the present disclosure.

FIG. 9 illustrates an interface representation depicting an integrated view of an invoice and one or more invoice entries, in accordance with an embodiment of the present disclosure.

FIG. 10 illustrates an interface representation depicting a user interface for creating anew invoice line entry within the invoice creation interface of the invoice management system, in accordance with an embodiment of the present disclosure.

FIGS. 11A and 11B illustrate interface representations depicting examples of invoicefee schedule configurations and corresponding invoice entry creation interfaces within the invoice management system, in accordance with an embodiment of the present disclosure.

FIGS. 12A, 12B, and 12C illustrate interface representations depicting examples oftraining service records and their associated data fields as displayed within invoice creation interface of the invoice management system, in accordance with an embodiment of the present disclosure.

FIGS. 13A and 13B collectively illustrate interface representations showing examples of finalized and approved invoices within the invoice management system, in accordance with an embodiment of the present disclosure.

FIG. 14 illustrates a method for generating an invoice for one or more tasks associated with a wearable medical device, in accordance with an embodiment of the present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, well-known structures or methods, associated with an authorization system for securing protected health information, have not been shown or described in detail to avoid unnecessarily obscuring descriptions of the embodiments.

Unless the context indicates otherwise, throughout the disclosure and claims which follow, the word "comprise" and variations thereof, such as, "comprises" and "comprising" are to be construed in an open, inclusive sense that is as "including, but not limited to." Further, the terms "first," "second," and similar indicators of the sequence are to be construed as interchangeable unless the context clearly dictates otherwise.

Reference throughout this disclosure to "one aspect" or "an aspect" means that a particular feature, structure, or characteristic described in connection with an embodiment is included in at least one aspect. Thus, the appearances of the phrases "in one aspect" or "in an aspect" in various places throughout this disclosure are not necessarily all referring to the same embodiment. Furthermore, the particular features, structures, or characteristics may be combined in any suitable manner in one or more aspects.

As used in this disclosure and the appended claims, the singular forms "a," "an," and "the" include plural referents unless the content clearly dictates otherwise. It should also be noted that the term "or" is generally employed in its broadest sense, that is, as meaning "and/or" unless the content clearly dictates otherwise.

In the interest of not obscuring the presentation of embodiments of the present disclosure, in the following detailed description, some processing steps or operations that are known in the art may have been combined together for presentation and for illustration purposes and in some instances may have not been described in detail. In other instances, some processing steps or operations that are known in the art may not be described at all. It should be understood that the following disclosure is rather focused on the distinctive features or elements of various embodiments of the present disclosure.

In enterprise environments involving distributed service personnel and contract-based medical device fitters, the generation, approval, and management of invoices often require integration between operational workflows and financial reporting systems. Embodiments of the present disclosure provide a configurable and dynamic invoice management system that facilitates automated invoice generation, task association, and approval tracking for activities related to servicing wearable medical devices.

Throughout the specification, the term "invoice management system" may refer to a computing platform, implemented on one or more processors and associated memory, configured to manage the creation, modification, approval, and storage of invoices. The invoice management system may include one or more graphical user interfaces such as an invoice creation interface, an invoice approval interface, and an administrator interface, each configured to enable respective functions within the invoicing workflow. The invoice management system may further include one or more data models, databases, or fee schedules storing invoice-related data, such as invoice entry options, subfields, and rate information.

Throughout the specification, the term "invoice creation interface" may refer to a graphical interface or user-accessible module of the invoice management system that enables a user, such as a contractor or fitter, to generate an invoice for one or more tasks associated with a wearable medical device. The invoice creation interface may be configured to receive a user creation input, such as a selection of an invoice type, and to dynamically display a plurality of invoice entry options based on the invoice type. The invoice creation interface may further receive one or more selected invoice entries corresponding to the invoice entry options and display one or more subfields associated with the selected invoice entries.

Throughout the specification, the term "invoice type" may refer to a classification ofan invoice according to a category of work or expense related to a wearable medical device. Examples of invoice types may include, without limitation, patient fitting services, training sessions, device retrievals, garment replacements, or expense reimbursements associated with such tasks. The invoice type may determine which invoice entry options are dynamically displayed to the user.

Throughout the specification, the term "invoice entry option" may refer to a selectable data record representing a task, service, or expense that may be billed on an invoice. Each invoice entry option may correspond to a predefined activity, such as "initial patient fitting," "training session," "follow-up visit," or "travel mileage." Each invoice entry option may be associated with one or more subfields for collecting additional information and with one or more corresponding entries in a fee schedule to determine applicable billing amounts.

Throughout the specification, the term "subfield" may refer to a data entry elemendynamically displayed within the invoice creation interface in response to the selection of a particular invoice entry option. Each subfield may require user input of information related to the selected task or expense, such as task descriptions, service dates, duration of work, mileage, item quantity, or related comments. The invoice management system may be configured to display different subfields for different invoice entry options, thereby allowing the user interface to adapt dynamically to the selected invoice type and associated data requirements.

Throughout the specification, the term "fee schedule" may refer to a data structurestored in the memory of the invoice management system that defines one or more fees, rates, or charge amounts corresponding to each invoice entry option. The fee schedule may include data fields identifying service types, applicable billing rates (flat or hourly), rate codes, and associated financial attributes. The fee schedule may be configurable via the administrator interface to add, remove, or modify entries without requiring redevelopment or hard-coded logic changes.

Throughout the specification, the term "administrator interface" may refer to agraphical interface or control module of the invoice management system that enables an authorized administrative user to configure, modify, or manage data structures used in invoice generation. The administrator interface may permit the addition or removal of invoice entry options, modification of fee schedule data, or definition of subfields for each invoice entry option.

Throughout the specification, the term "invoice approval interface" may refer to agraphical interface of the invoice management system that enables a supervisory user or finance personnel to review and approve submitted invoices. The invoice approval interface may be configured to update an invoice status among one or more predefined values such as "draft," "submitted," "approved," or "rejected." The invoice approval interface may also trigger automatic generation of a status communication and a final invoice form upon approval.

Throughout the specification, the term "status communication" may refer to anautomatically generated message transmitted by the invoice management system to one or more users when the status of an invoice changes. The status communication may include, for example, an email notification, application alert, or data message indicating that the invoice has been submitted, approved, or rejected.

Throughout the specification, the term "work order" may refer to a record generated in response to a request to perform one or more tasks related to a wearable medical device. The work order may include identifiers for a patient, a medical device, and a fitter or contractor assigned to perform the associated service. The work order may be used to automatically populate or pre-fill one or more invoice entries when an invoice is created, thereby maintaining consistency between service performance and billing.

Throughout the specification, the term "wearable medical device" may refer to any therapeutic or monitoring apparatus designed to be worn by a patient and requiring fitting, adjustment, or training for proper operation. Examples of wearable medical devices may include defibrillator vests, monitoring garments, or other therapeutic wearables provided under medical prescription.

Throughout the specification, the term "contractor computing device" may refer to a user device, such as a laptop computer, tablet, or mobile device, used by a fitter or contractor to access the invoice management system, receive work orders, and generate invoices. The contractor computing device may communicate with the invoice management system over a network connection and may host a local client application or web interface for data entry and submission.

In various embodiments, the present disclosure enables dynamic invoice generationand approval management through a configurable invoice management system that integrates task- related data, contextual fee determination, and adaptive user interfaces. The invoice management system operates as a unified digital framework wherein a contractor computing device interacts with an invoice creation interface to generate invoices corresponding to one or more tasks associated with a wearable medical device. The invoice creation interface dynamically adapts its display based on a selected invoice type to present relevant invoice entry options and subfields, each linked to a fee defined in a fee schedule of the invoice management system. The system automatically computes applicable charges in real time, thereby decreasing manual input and calculation errors. In some embodiments, the invoice management system further retrieves work order information to prepopulate invoice entries corresponding to completed service activities such as patient fitting or device training. An administrator interface provides configurable control to add, remove, or modify invoice entry options, subfields, or fee structures without requiring back- end reprogramming.

The invoice approval interface enables review, approval, and rejection workflows, and the system automatically issues status communications to relevant users upon any change in invoice status. Upon approval, the invoice management system generates a consolidated invoice form comprising the selected invoice entries, their associated fees, and approval metadata. The system may further integrate with external enterprise resource planning or billing platforms to enable seamless data synchronization, audit traceability, and secure record exchange across organizational boundaries, thereby improving financial accuracy, operational efficiency, and transparency in enterprise invoicing workflows for wearable medical device servicing.

The present disclosure will now be described in detail with reference to the Figures.

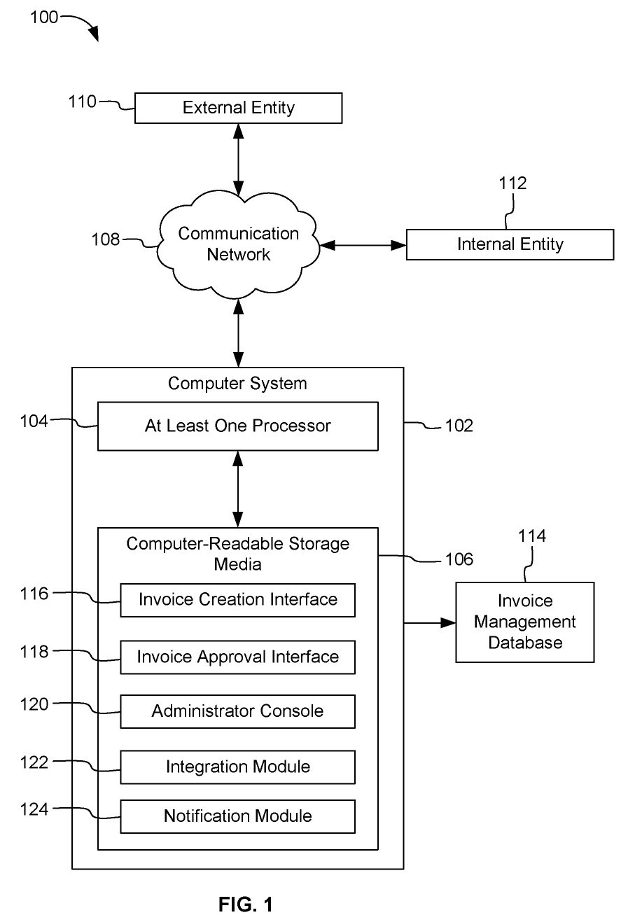

With reference now to FIG. 1, a computer environment 100 for generating andmanaging invoices related to servicing a wearable medical device for a patient is illustrated, in accordance with an embodiment of the present disclosure. The computer environment 100 includes a computer system 102, at least one processor 104, computer-readable storage media 106, a communication network 108, an external entity 110, an internal entity 112, an invoice management database 114, an invoice creation interface 116, an invoice approval interface 118, an administrator console 120, an integration module 122, and a notification module 124. The computer system 102 serves as a central computing infrastructure that orchestrates interactions among the various components of the computer environment 100 through the communication network 108.

Within the context of the present disclosure, the term "computer environment" mayrefer to an integrated software and hardware ecosystem configured to facilitate creation, management, review, approval, and communication of invoices associated with services performed on wearable medical devices. The computer environment 100 may be deployed in an enterprise network, a hospital information system, or a cloud-based platform that supports distributed users such as contractors, healthcare providers, and administrative personnel. In an embodiment, the computer environment 100 enables automation of invoice lifecycle events, dynamic configuration of invoice elements, and real-time coordination between external service contractors and internal organizational reviewers.

In some embodiments, the computer system 102 functions as the core processing node within the computer environment 100. The computer system 102 may be implemented as a server computer, a cluster of virtualized servers, or a cloud service instance provisioned within a healthcare or enterprise domain. The computer system 102 is communicatively coupled with the communication network 108 to exchange data with the external entity 110 and the internal entity 112. The computer system 102 includes the at least one processor 104 and the computer-readable storage media 106, which collectively execute program modules and store operational data required to perform the operations described herein.

The at least one processor 104 may include one or more central processing units (CPUs), graphics processing units (GPUs), or any hardware accelerator configured to execute software instructions stored on the computer-readable storage media 106. The at least one processor 104 is configured to receive, process, and respond to user creation inputs originating from the external entity 110 through the invoice creation interface 116. The at least one processor 104 is further configured to dynamically display a plurality of invoice entry options based on an invoice type selected by the external entity 110, where the plurality of invoice entry options are associated with one or more tasks and/or one or more expenses related to servicing the wearable medical device. In addition, the at least one processor 104 is configured to determine, through reference to a fee schedule stored within the invoice management database 114, a fee corresponding to each of the selected invoice entries and to generate an invoice including the selected invoice entries and the associated fees.

The external entity 110 may be a contractor, a vendor, or a third-party service provider responsible for performing one or more tasks associated with fitting or maintaining a wearable medical device or training a patient in the use thereof. The external entity 110 interacts with the invoice creation interface 116 hosted by the computer system 102 to create the invoice following completion of the one or more tasks. The external entity 110 may provide a user creation input specifying an invoice type, such as, but not limited to, "device fitting," "training session," "routine maintenance," or "hardware replacement." Upon receiving the user creation input, the at least one processor 104 dynamically renders corresponding invoice entry options such as service duration, device category, consumable items, or travel cost.

In an embodiment, subsequent to receiving one or more selected invoice entries, the at least one processor 104 is configured to dynamically display a plurality of subfields corresponding to the selected invoice entries. Each invoice entry option may have unique subfields, for example, a service task entry may include subfields for any of, but not limited to, "task description," "device identifier," or "time spent," whereas an expense entry may include subfields for "expense type," "amount," and "receipt upload." The at least one processor 104 receives user subfield inputs through the invoice creation interface 116, thereby capturing detailed information associated with each of the selected invoice entries.

In another embodiment, the external entity 110 may initially receive, on a contractor computing device, a service request related to the wearable medical device. Upon accepting the service request, the at least one processor 104 generates a corresponding work order stored in the invoice management database 114. The work order may record the patient identifier, device serial number, service type, and completion status. The at least one processor 104 may automatically link the generated invoice to the corresponding work order such that any completed task or approved service is reflected as an auto-populated invoice entry. In this manner, the at least one processor 104 automates invoice generation directly from operational work data, thereby reducing manual entry and improving accuracy.

The external entity 110 operates through a contractor computing device that communicates with the computer system 102 via the communication network 108. The contractor computing device may be a mobile device, a tablet, or a web-enabled workstation used by field personnel providing patient services. The at least one processor 104 authenticates the external entity 110 on the contractor computing device and provides access to assigned work orders, service requests, and invoice creation features through a unified interface.

In some embodiments, prior to receiving the user creation input, the at least oneprocessor 104 is configured to receive, at a contractor computing device associated with the external entity 110, a request to perform one or more tasks related to a wearable medical device for a patient. The request may originate from an internal scheduling system, a healthcare provider portal, or a patient management platform integrated through the integration module 122. Upon receipt of the request, the at least one processor 104 enables the external entity 110 to accept the request via the contractor computing device, which in turn generates a corresponding work order stored within the invoice management database 114. The work order represents a structured record including identifiers for the patient, the wearable medical device, task descriptions, assigned contractor details, and expected service completion timelines.

The work order is associated with the invoice such that each task or expense identifiedin the work order maps to a corresponding invoice entry option displayed through the invoice creation interface 116. The at least one processor 104 is further configured to automatically add one or more invoice entries to the invoice and/or automatically populating one or more invoice entries based on the work order, including pre-defined fees and service descriptions retrieved from the fee schedule of the invoice management database 114. The automatic population of invoice data reduces manual effort and ensures alignment between performed tasks and invoiced items.

In an exemplary embodiment, the external entity 110 performs the one or more tasks associated with the wearable medical device following acceptance of the work order. The one or more tasks may include, but are not limited to, fitting the patient with the wearable medical device, calibrating the device for operational accuracy, or training the patient in the correct usage and maintenance of the device. Upon completion of the one or more tasks, the at least one processor 104 records service completion data in the invoice management database 114, enabling the subsequent generation of an accurate and verified invoice through the invoice creation interface 116.

In some embodiments, the internal entity 112 represents authorized personnel within an organization, such as finance or operations departments, who are responsible for reviewing, approving, or rejecting invoices submitted by the external entity 110. The internal entity 112 interacts with the invoice approval interface 118 hosted by the computer system 102. The at least one processor 104 is configured to enable the internal entity 112 to review submitted invoices, verify the details captured through subfields, and approve or reject invoices in accordance with organizational policy or contractual terms. The at least one processor 104 further records the approval decision in the invoice management database 114.

The invoice management database 114 serves as a structured data repositoryconfigured to persist invoices, work orders, fee schedules, user profiles, and system configuration data. The invoice management database 114 may be implemented as a relational or non-relational data store that maintains data integrity and auditability. The at least one processor 104 interacts with the invoice management database 114 to retrieve applicable fee schedules based on service category, contractor role, or regional pricing policies, and to compute total amounts for each generated invoice. The at least one processor 104 further stores generated invoices in the invoice management database 114, along with metadata such as invoice creation timestamp, status, and user identifiers.

The administrator console 120 provides an interface for authorized administrators to configure operational parameters of the invoice management system. The at least one processor 104 is configured to enable an administrator, through the administrator console 120, to add or remove invoice entry options, define or modify subfields associated with each invoice entry option, and update fee schedules maintained in the invoice management database 114. The administrator console 120 allows flexibility for adapting the system 102 to different service models, device categories, or regional requirements without redeploying application code.

The integration module 122 facilitates communication between the computer system 102 and external enterprise systems. The integration module 122 may include APIs or middleware connectors enabling secure data exchange with systems such as enterprise resource planning (ERP), accounting, or customer relationship management (CRM) platforms. The at least one processor 104 utilizes the integration module 122 to transmit approved invoices to an external accounting system for payment processing or to receive customer information, billing addresses, and purchase order references from an external CRM. The integration module 122 ensures synchronization of invoice status and financial records across organizational systems.

The notification module 124 is configured to automatically generate and send communications related to invoice status changes. The at least one processor 104, in conjunction with the notification module 124, may automatically transmit electronic notifications such as emails, system alerts, or mobile push messages to the external entity 110 or the internal entity 112 upon occurrence of predefined status events, including "submitted," "approved," or "rejected." The notification module 124 thus maintains transparency of invoice processing and enhances workflow responsiveness.

In some embodiments, upon a change in the status of an invoice, the at least one processor 104, in cooperation with the notification module 124, automatically sends a status communication to relevant users. The users may include the external entity 110, the internal entity 112, and administrative personnel accessing the administrator console 120. The communication may include one or more of an email notification, an in-system message, or a push alert through an enterprise application. The status communicated includes, but is not limited to, "submitted," "approved," and "rejected." The notification module 124 ensures real-time transparency across all stakeholders regarding the progress of invoice review and approval within the computer environment 100.

The communication network 108 provides the data transmission backbone of the computer environment 100. The communication network 108 may include one or more wired or wireless networks employing protocols such as Transmission Control Protocol/Internet Protocol (TCP/IP), Hyper Text Transfer Protocol (HTTP), Hyper Text Transfer Protocol Secure (HTTPS), or secure WebSocket connections. In some embodiments, the communication network 108 ensures encrypted and authenticated communication between the computer system 102, the external entity 110, and the internal entity 112. The communication network 108 may further support cloud-based access, enabling geographically distributed users to securely interact with the computer environment 100.

In an exemplary operation, the at least one processor 104 orchestrates the functional interaction among all components of the computer environment 100. For example, upon receiving a service completion notification from the external entity 110, the at least one processor 104 retrieves applicable fee data from the invoice management database 114, dynamically populates invoice fields through the invoice creation interface 116, and records the resulting invoice for review. The at least one processor 104 then enables the internal entity 112 to access the invoice approval interface 118 to approve or reject the invoice. Upon approval, the at least one processor 104 triggers the notification module 124 to send confirmation messages and uses the integration module 122 to forward the approved invoice to the external accounting system for payment.

Upon approval of an invoice by the internal entity 112, the at least one processor 104is configured to generate a consolidated invoice form. The consolidated invoice form includes the one or more selected invoice entries, the fee for each of the one or more selected invoice entries as determined from the fee schedule of the invoice management database 114, and the current status of the invoice. The consolidated invoice form may further include additional metadata such as approval timestamp, approver identification, and payment references. The at least one processor 104 stores the consolidated invoice form within the invoice management database 114 and may transmit the consolidated invoice form to external systems via the integration module 122 for payment processing, financial auditing, or record retention. The consolidated invoice form serves as an official and immutable record of the approved invoice transaction within the computer environment 100.

In an alternate embodiment, the computer-readable storage media 106 may store additional modules, including a validation engine configured to verify completeness of invoice subfield data before submission, and a reporting engine configured to generate summaries or analytics across multiple invoices. The at least one processor 104 may execute the validation engine to detect missing or inconsistent entries and prompt the external entity 110 for corrections through the invoice creation interface 116. In some embodiments, prior to generating the invoice, the validation engine may detect the inconsistencies in one or more invoice entries or subfields based on at least one of the work order or one more additional supporting documents. The at least one processor 104 may execute the reporting engine to compile invoice statistics, performance metrics, or audit trails accessible to administrators through the administrator console 120.

In an exemplary scenario, the external entity 110, such as a contractor performing device fitting for a patient, accesses the invoice creation interface 116 to select the invoice type "device fitting." The at least one processor 104 retrieves predefined entry options such as "fitting session time," "travel distance," and "consumables used." The external entity 110 inputs corresponding data into dynamically displayed subfields and submits the invoice. The at least one processor 104 computes fees based on the stored fee schedule, generates a total invoice amount, and stores the invoice in the invoice management database 114. The internal entity 112 reviews the invoice through the invoice approval interface 118 and approves it. Upon approval, the at least one processor 104 automatically updates the invoice status, triggers the notification module 124 to inform the external entity 110, and exports the approved invoice via the integration module 122 to an accounting platform for payment processing.

In another exemplary scenario, the computer environment 100 may be deployed within a healthcare service provider's network. The external entity 110 represents contracted field technicians responsible for wearable device servicing, while the internal entity 112 represents hospital billing personnel. The at least one processor 104 dynamically enforces invoice standardization and traceability across multiple contractors, ensuring that all invoices adhere to configurable fee schedules and contain complete subfield information. Such automation reduces administrative overhead and ensures regulatory compliance for medical billing workflows.

The modular architecture of the computer environment 100 allows for scalability andadaptability. Each interface or module, namely the invoice creation interface 116, the invoice approval interface 118, the administrator console 120, the integration module 122, and the notification module 124, may be implemented as an independent microservice communicating with the computer system 102 through standardized APIs. The at least one processor 104 coordinates inter-module communication, thereby supporting efficient load balancing, fault tolerance, and secure data integrity across all invoice management operations.

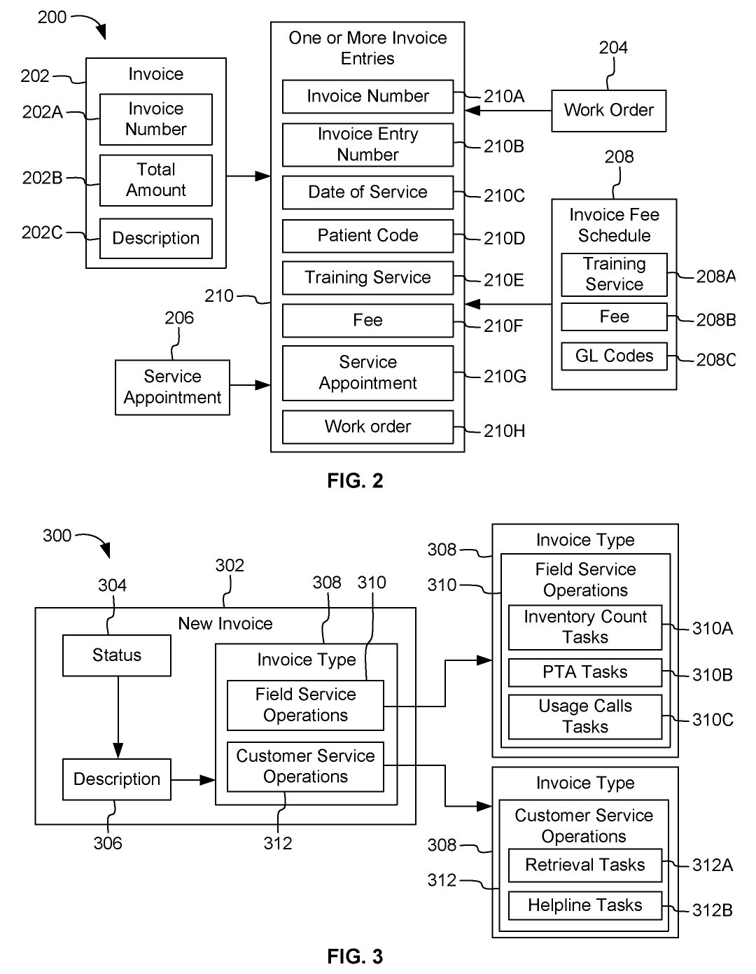

FIG. 2 illustrates a data model 200 depicting the interaction between various datacomponents and entities during invoice generation, processing, and approval within the computer environment 100, in accordance with one or more embodiments of the present disclosure. FIG. 2 is described in conjunction with FIG. 1. The data model 200 represents the logical structure of the invoice management database 114 and its integration with related operational entities such as work orders, service appointments, and fee schedules. The at least one processor 104 may be configured to coordinate the flow of data between the data components, ensuring that the invoice data remains consistent, accurate, and dynamically updated throughout the lifecycle of invoice creation and approval.

The data model 200 includes an invoice 202, a work order 204, a service appointment 206, and an invoice fee schedule 208. Each of the invoice 202, the work order 204, the service appointment 206, and the invoice fee schedule 208 contribute as structured data that is combined to form one or more invoice entries 210. The one or more invoice entries 210 are stored within the invoice management database 114 and are processed through the invoice creation interface 116 for user interaction. The at least one processor 104 is configured to maintain referential integrity among the components of the data model 200, ensuring that every invoice entry of the one or more invoice entries 210 correctly references a corresponding invoice such as the invoice 202, the work order 204, and the service appointment 206.

The invoice 202 includes one or more key data fields such as an invoice number 202a, a total amount 202b, and a description 202c. The invoice number 202a uniquely identifies each invoice within the invoice management database 114, ensuring traceability and version control during review and approval. The total amount 202b represents the computed aggregate of all fees associated with the one or more invoice entries 210 linked to the invoice 202. The description 202c provides contextual information summarizing the nature of the invoice 202. In an example, the contextual information may be in the form as "Device Fitting and Patient Training, October 2024." The at least one processor 104 may automatically update the total amount 202b based on recalculations performed when any invoice entry of the one or more invoice entries 210 linked to the invoice 202 is modified.

The work order 204 stores information corresponding to one or more tasks or services performed by the external entity 110 related to a wearable medical device. The work order 204 may be generated when the contractor computing device of the external entity 110 receives and accepts a request to perform the one or more tasks associated with a patient. The work order 204 may include details such as, but not limited to, task identifiers, task descriptions, assigned technician information, service duration, and completion status. The work order 204 is directly associated with the invoice 202 through a unique work order identifier, enabling the at least one processor 104 to automatically populate the one or more invoice entries 210 based on completed tasks recorded in the work order 204. In an example, when a technician completes a wearable medical device fitting for a patient, the at least one processor 104 may automatically generate a corresponding invoice entry of the one or more invoice entries 210 populated with task details, a date of service, and applicable fee information retrieved from the invoice fee schedule 208.

The service appointment 206 captures the temporal and logistical aspects of the oneor more tasks performed under the work order 204. The service appointment 206 may include a scheduled date, a service location, a patient identification code, an assigned technician, and a confirmation of service completion. The service appointment 206 links to the work order 204 and the invoice 202, enabling synchronization between task execution and invoice generation. The at least one processor 104 may validate that the service appointment 206 has a confirmed completion status before generating the invoice 202 to ensure accurate billing. In an example, when the service appointment 206 corresponds to a training session for a patient on the use of a wearable medical device, the at least one processor 104 ensures that the service appointment 206 has been marked as "Completed" prior to generating the corresponding invoice entry of the one or more invoice entries 210.

The invoice fee schedule 208 defines standardized billing structures for numerous services associated with the wearable medical device. The invoice fee schedule 208 includes one or more fields such as a training service 208a, a fee 208b, and a general ledger (GL) code 208c. The GL code 208c represents an accounting identifier used to classify revenue or expense categories within an enterprise resource planning system. When the external entity 110 selects a specific service type through the invoice creation interface 116, the at least one processor 104 references the invoice fee schedule 208 to retrieve the appropriate fee 208b and the GL code 208c corresponding to the selected service. The training service 208a may include predefined entries such as "Patient Device Setup," "Follow-Up Calibration," or "Usage Refresher Session." In some embodiments, the at least one processor 104 may dynamically adjust the fee 208b based on modifiers such as service location, service duration, or urgency parameters defined within the invoice fee schedule 208.

The one or more invoice entries 210 consolidate data from the invoice 202, the work order 204, the service appointment 206, and the invoice fee schedule 208. Each invoice entry of the one or more invoice entries 210 includes multiple fields, including but not limited to an invoice number 210a, an invoice entry number 210b, a date of service 210c, a patient code 210d, a training service 210e, a fee 210f, a service appointment 210g, and a work order 210h. Each invoice entry of the one or more invoice entries 210 represents an individual billable activity linked to a specific task performed under the work order 204. The at least one processor 104 is configured to automatically populate each invoice entry of the one or more invoice entries 210 based on data retrieved from the invoice management database 114, ensuring that every field in each invoice entry of the one or more invoice entries 210 maintains consistency with the corresponding source record.

In an exemplary embodiment, when the external entity 110 initiates creation of the invoice 202 through the invoice creation interface 116, the at least one processor 104 retrieves all completed work orders such as the work order 204 and all corresponding service appointments such as the service appointment 206 for a defined period. The at least one processor 104 dynamically displays one or more available invoice entry options to the external entity 110, each corresponding to a potential invoice entry of the one or more invoice entries 210. For example, when a contractor has performed three training sessions and one fitting service during a defined time period, the invoice creation interface 116 displays four selectable invoice entry options, each linked to a task description, the patient code 210d, and the fee 210f. Upon selection of invoice entries options, the at least one processor 104 retrieves associated fee data 208b and the GL code data 208c from the invoice fee schedule 208 and populates each corresponding invoice entry of the one or more invoice entries 210 accordingly.

The external entity 110 may enter additional contextual information within each invoice entry of the one or more invoice entries 210 by populating subfields dynamically displayed by the invoice creation interface 116. The one or more subfields may include supplementary notes, mileage expenses, or time-based adjustments. The at least one processor 104 validates each subfield entry and updates the total amount 202b of the invoice 202 to reflect any modifications in the fee 210f. For example, when the external entity 110 adds an expense related to travel during patient training, the at least one processor 104 retrieves the applicable travel fee from the invoice fee schedule 208 and applies the travel fee to the total amount 202b while preserving the audit trail of the invoice 202 within the invoice management database 114.

When the invoice 202 and the one or more invoice entries 210 are finalized, the at least one processor 104 enables the internal entity 112 to review and approve the invoice 202 through the invoice approval interface 118. The invoice approval interface 118 displays all fields of the invoice 202 and all fields of the one or more invoice entries 210 in a consolidated layout, including corresponding references to the work order 204 and the service appointment 206. Upon approval of the invoice 202, the at least one processor 104 generates a consolidated invoice form including the invoice number 202a, the total amount 202b, details of the one or more invoice entries 210, and approval metadata. The consolidated invoice form is stored within the invoice management database 114 and transmitted to an external accounting system through the integration module 122 for payment processing.

In an exemplary use case, the external entity 110 completes a wearable medical device fitting and two training sessions during a single patient visit. The at least one processor 104 retrieves the data from the work order 204 and the service appointment 206 corresponding to the completed tasks. The invoice creation interface 116 dynamically displays three invoice entry options corresponding to three invoice entries of the one or more invoice entries 210, each representing a completed service. The external entity 110 selects all three invoice entry options, and the at least one processor 104 references the invoice fee schedule 208 to fetch the corresponding fees 208b and GL codes 208c. The invoice 202 is generated with the total amount 202b computed in real time. The internal entity 112 reviews the invoice 202 through the invoice approval interface 118, approves the invoice 202, and the at least one processor 104 issues a status communication through the notification module 124 indicating that the invoice 202 is "Approved." The invoice management database 114 stores the finalized invoice 202 and the one or more invoice entries 210, ensuring complete traceability and transparency in the billing workflow.

Accordingly, FIG. 2 provides a structured representation of the data relationships, dependencies, and automated interactions that occur within the invoice management system. The data model 200 supports dynamic generation, validation, and approval of the invoice 202 and the one or more invoice entries 210 related to servicing wearable medical devices. The configuration of the data model 200 ensures accurate fee computation, traceable linkage between operational and financial data, and efficient automation of enterprise billing workflows.

FIG. 3 illustrates an interface representation 300 depicting a graphical layout of a newinvoice 302 as displayed within the invoice creation interface 116 of an invoice managementsystem, in accordance with one or more embodiments of the present disclosure. FIG. 3 is described in conjunction with FIGS. 1 and 2. The interface representation 300 visually demonstrates how the at least one processor 104 dynamically generates and renders one or more data input fields for creating the invoice 202 and the one or more invoice entries 210 within the invoice management database 114. The interface representation 300 enables the external entity 110 to enter or select information for initializing the invoice 202, including a status 304, a description 306, and an invoice type 308.

In some embodiments, the new invoice 302 represents an initial record instance created by the external entity 110 to begin the invoice generation process. The status 304 indicates the current state of the invoice 202, such as "New," "In Progress," "Submitted," or "Approved." The at least one processor 104 automatically assigns the status 304 as "New" when the external entity 110 initiates creation of the new invoice 302, thereby ensuring version control and traceability across all invoice stages. The description 306 provides contextual labeling for the invoice 202, for example, "July 1-15 Service Period," "Quarterly Device Fittings," or other textual identifiers that specify the billing cycle or project reference. The at least one processor 104 stores the status 304 and the description 306 within the invoice management database 114 to maintain consistent metadata across related invoice entries 210.

The invoice type 308 field within the interface representation 300 allows the externalentity 110 to specify the operational category of the invoice 202. The at least one processor 104 dynamically expands the invoice type 308 to display multiple selectable categories corresponding to service domains managed by the invoice management system. In some embodiments, the invoice type 308 includes two primary categories: field service operations 310 and customer service operations 312. Each category includes predefined task types relevant to its operational context. The at least one processor 104 retrieves configuration data defining each category and associated task types from the invoice management database 114 and dynamically renders the retrieved configuration within the invoice creation interface 116.

The selection of the field service operations 310 category by the external entity 110prompts the at least one processor 104 to expand the invoice type 308 and display corresponding task types including an inventory count task 310a, a Patient Training Agreement (PTA) task 310b, and a usage calls task 310c. Each task type represents a discrete operational activity related to the maintenance or deployment of the wearable medical devices. The inventory count task 310a may correspond to activities such as verifying available device quantities at patient or clinic locations. The PTA task 310b may correspond to fitting and instructional sessions for patients learning to operate wearable medical devices. The usage calls task 310c may correspond to periodic follow- up interactions verifying device functionality or patient compliance. When any task type of the field service operations 310 is selected, the at least one processor 104 generates one or more invoice entries 210 pre-populated with identifiers, fee structures, and general ledger (GL) codes 208c retrieved from the invoice fee schedule 208.

Similarly, selection of the customer service operations 312 category causes the at leastone processor 104 to expand the invoice type 308 to reveal task types including a retrieval task 312a and a helpline task 312b. The retrieval task 312a may relate to collection or replacement of wearable medical devices from patients, while the helpline task 312b may correspond to remote support or troubleshooting services provided by the external entity 110. The at least one processor 104 ensures that selection of each task type within the customer service operations 312 automatically references corresponding records within the invoice fee schedule 208, thereby ensuring accurate fee determination and linkage of all accounting identifiers.

In some embodiments, the at least one processor 104 monitors selections made within the invoice type 308 in real time, dynamically adjusting the user interface of the invoice creation interface 116 to display only those subfields relevant to the chosen operational category and associated task types. For example, when the external entity 110 selects the field service operations 310 category, additional subfields for patient codes, appointment identifiers, and service locations may be displayed, whereas selection of the customer service operations 312 category may prompt display of communication channel identifiers and resolution notes. The at least one processor 104 validates each input received in response to these dynamically generated subfields and ensures consistency with stored definitions in the invoice management database 114.

The interface representation 300 therefore provides a structured and guided workflowthat ensures uniformity in how invoices are created and categorized. By dynamically displaying only those options and task types that are relevant to the selected operational category, the invoice management system reduces the likelihood of data entry errors and eliminates the need for manual cross-referencing. The at least one processor 104, in coordination with the communication network 108 and the invoice management database 114, synchronizes all selections made through the interface representation 300 with corresponding records in the invoice 202, the work order 204, and the service appointment 206.

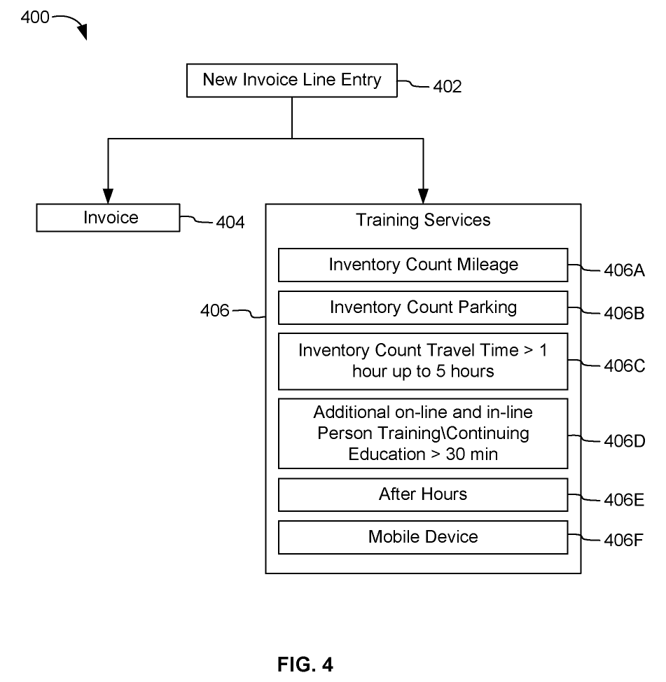

FIG. 4 illustrates an interface representation 400 depicting creation of a new invoice line entry 402 within the invoice creation interface 116 of the invoice management system, in accordance with one or more embodiments of the present disclosure. FIG. 4 is described in conjunction with FIGS. 1-3. The interface representation 400 represents the detailed configuration view where the external entity 110 enters specific billing components that form part of the one or more invoice entries 210 corresponding to an invoice 404. The at least one processor 104 is configured to dynamically render and manage multiple input subfields within the invoice line entry 402, each subfield associated with a particular service or expense type retrieved from the invoice fee schedule 208.

The invoice 404 displayed within the interface representation 400 includes a unique invoice identifier, for example, "INV-202501-00699," automatically assigned and managed by the at least one processor 104. The unique invoice identifier enables the invoice 404 to be correlated with corresponding operational records in the work order 204 and service appointment 206 stored within the invoice management database 114. The at least one processor 104 ensures that every invoice line entry 210 created within the invoice 404 is properly linked to this identifier, preserving end-to-end traceability from service execution to billing.

The training services 406 section of the interface representation 400 provides anoperational grouping of selectable subfields related to patient training, technician support, and travel-based activities associated with the wearable medical device. In some embodiments, the training services 406 include an inventory count mileage 406a, an inventory count parking 406b, an inventory count travel time 406c, an additional on-line and in-line person training 406d, after hours 406e, and a mobile device 406f (such as ASSURE mobile device). Each subfield corresponds to a distinct component of the service effort that may contribute to the total invoice fee. The at least one processor 104 retrieves the applicable rates, fee codes, and general ledger (GL) codes 208c for each subfield from the invoice fee schedule 208, ensuring accurate and automatic cost allocation.

When the external entity 110 initiates creation of the new invoice line entry 402, the at least one processor 104 dynamically displays the training services 406 and associated subfields based on the invoice type 308 previously selected in the interface representation 300 of FIG. 3. For example, if the invoice type 308 corresponds to field service operations 310, the training services 406 may automatically populate with subfields corresponding to travel and patient training activities, such as the inventory count mileage 406a, the inventory count travel time 406c, and the additional on-line and in-line person training 406d. Each selection made by the external entity 110 in the training services 406 generates a corresponding invoice line entry 210 with metadata linking to the work order 204 and service appointment 206.

The at least one processor 104 computes the fee associated with each subfield of thetraining services 406 using predefined formulas or parameters stored in the invoice fee schedule 208. For instance, the inventory count mileage 406a may be calculated using a distance-based rate multiplied by the total miles recorded during service, while the inventory count parking 406b may be retrieved as a fixed reimbursement charge. The after hours 406e subfield may apply a surcharge multiplier to the base service rate to reflect off-schedule labour. The ASSURE mobile device 406f may correspond to a specific device training activity or software configuration fee related to the wearable medical device provided to the patient. All calculated fees are automatically aggregated by the at least one processor 104 to determine a total charge for the new invoice line entry 402.

The invoice creation interface 116 enables the external entity 110 to review andconfirm each invoice line entry 210 generated from the selected subfields of the training services 406. Upon confirmation, the at least one processor 104 commits the data to the invoice management database 114, tagging each invoice line entry 210 with identifiers of the invoice 404, the work order 204, and the service appointment 206. The above-described linkage ensures that each billing component is auditable and verifiable against operational activities recorded in the invoice management system. The communication network 108 facilitates synchronization between the contractor computing device of the external entity 110 and the at least one processor 104 executing the invoice management system, ensuring that the new invoice line entry 402 is consistently reflected across all connected enterprise systems.

In some embodiments, the at least one processor 104 may automatically generate the one or more invoice entries 210 based on data contained in the work order 204, thereby enabling partial automation of the invoice creation process. In an example, when a work order 204 indicates that a patient fitting and device setup task was completed, the at least one processor 104 automatically populates the training services 406 with corresponding subfields, such as the additional on-line and in-line person training 406d, and assigns the fee from the invoice fee schedule 208. The external entity 110 may then verify and adjust the automatically generated entries prior to submitting the invoice 404 for approval.

Accordingly, FIG. 4 illustrates the operational layer of the invoice creation interface 116 where detailed, task-specific billing data is captured, validated, and linked to corresponding operational records. The structured arrangement of the invoice 404 and the training services 406 subfields, including the inventory count mileage 406a, the inventory count parking 406b, the inventory count travel time 406c, the additional on-line and in-line person training 406d, the after hours 406e, and the ASSURE mobile device 406f, ensures that the invoice management system produces precise, traceable, and contextually relevant invoice entries 210. The dynamic subfield presentation, real-time fee computation, and automated record linkage collectively enhance the efficiency, accuracy, and transparency of enterprise invoice generation for wearable medical device servicing.

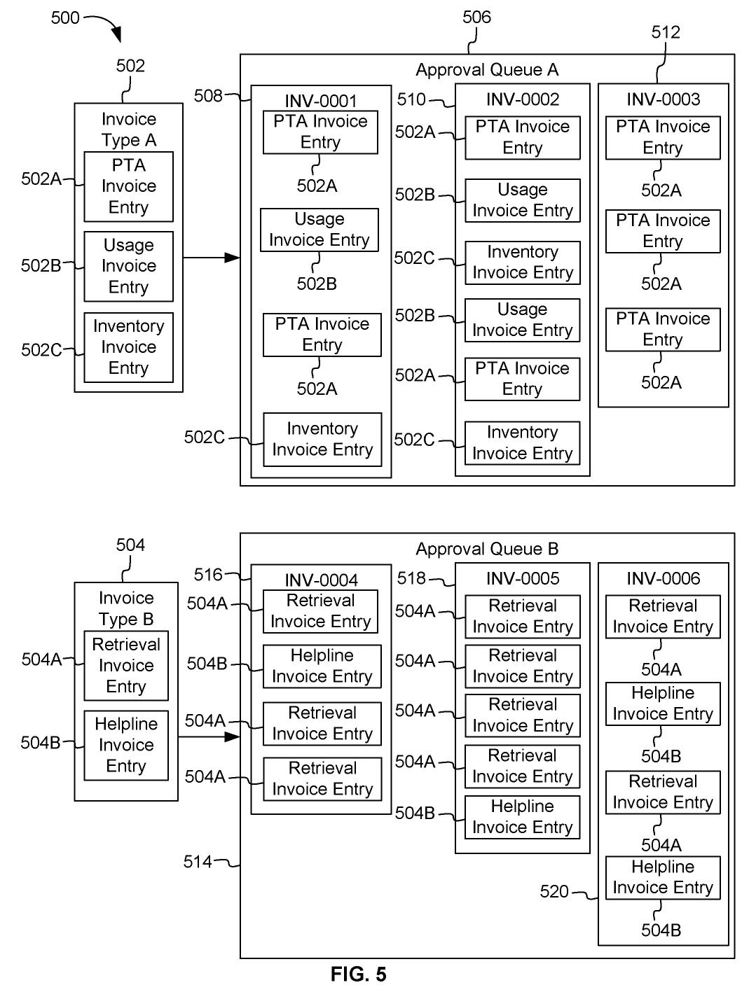

FIG. 5 illustrates a workflow diagram 500 representing routing and approval of different invoice types and their corresponding invoice entries within the invoice management system, in accordance with one or more embodiments of the present disclosure. FIG. 5 is described in conjunction with FIGS. 1-4. The workflow diagram 500 demonstrates how the at least one processor 104 and the computer-readable storage media 106 cooperate to organize, route, and manage approval of invoices generated through the invoice creation interface 116 based on their invoice type 308. The workflow diagram 500 reflects the logical structure implemented by the invoice management database 114 and the approval interface 118, enabling automated segregation of invoices, systematic queuing for approval, and secure validation by designated internal entity 112.

The workflow diagram 500 includes an invoice type A 502 and an invoice type B 504, each representing a distinct category of invoices based on the nature of service operations. The invoice type A 502 corresponds to field service operations 310 and includes one or more invoice entries 502a-502c such as a Patient Training Agreement (PTA) invoice entry 502a, a usage invoice entry 502b, and an inventory invoice entry 502c. The invoice type B 504 corresponds to customer service operations 312 and includes one or more invoice entries 504a-504b, such as a retrieval invoice entry 504a and a helpline invoice entry 504b. Each invoice entry of the invoice type A 502 or the invoice type B 504 represents a detailed billing element generated in the invoice entry table 210 and stored within the invoice management database 114 as described previously with reference to FIG. 2.

Upon creation and validation of the one or more invoice entries 502a-502c and invoice entries 504a-504b, the at least one processor 104 automatically classifies each invoice 404 into a corresponding approval queue, such as an approval queue A 506 or an approval queue B 514, based on the invoice type to which the invoice 404 belongs. The invoice type A 502 is routed to the approval queue A 506, while the invoice type B 504 is routed to the approval queue B 514. This classification and routing are performed by the at least one processor 104, which references preconfigured routing logic stored in the invoice management database 114. The at least one processor 104 ensures that every invoice 404 assigned to an approval queue maintains linkage integrity with the relevant invoice type and invoice entries.

The approval queue A 506 includes a plurality of invoices, for example, a first invoice 508, a second invoice 510, and a third invoice 512. Each of the first invoice 508, the second invoice 510, and the third invoice 512 includes one or more invoice entries 502a-502c corresponding to the invoice type A 502. In an example, the first invoice 508 may include four invoice entries, two corresponding to the PTA invoice entry 502a, one corresponding to the usage invoice entry 502b, and one corresponding to the inventory invoice entry 502c. The second invoice 510 may include six invoice entries, two each of the PTA invoice entry 502a, the usage invoice entry 502b, and the inventory invoice entry 502c. Similarly, the third invoice 512 may include three invoice entries, each corresponding to the PTA invoice entry 502a. The at least one processor 104 retrieves, validates, and aggregates the financial data of these invoice entries 502a-502c from the invoice management database 114 to compute the total charge for each invoice 404 routed into the approval queue A 506.

In some embodiments, the invoice type B 504 is directed by the at least one processor 104 to the approval queue B 514. The approval queue B 514 includes a fourth invoice 516, a fifth invoice 518, and a sixth invoice 520. Each of these invoices includes one or more invoice entries 504a-504b associated with the invoice type B 504. In one example, the fourth invoice 516 may include four invoice entries, three corresponding to the retrieval invoice entry 504a and one corresponding to the helpline invoice entry 504b. The fifth invoice 518 may include five invoice entries, four corresponding to the retrieval invoice entries 504a and one corresponding to the helpline invoice entry 504b. Similarly, the sixth invoice 520 may include four invoice entries, two corresponding to the retrieval invoice entry 504a and two corresponding to the helpline invoice entry 504b. The at least one processor 104 ensures that each of the invoice entries 504a-504b retains metadata linking to the corresponding work order 204 and service appointment 206 for audit and compliance verification.

Once the invoice 404 is routed into the appropriate approval queues (506 or 514), the internal entity 112 designated for the corresponding invoice type accesses the queue through the approval interface 118. In the approval queue A 506, an authorized member of the internal entity 112 may review each PTA invoice entry 502a, usage invoice entry 502b, and inventory invoice entry 502c associated with the invoice 404. The internal entity 112 validates each invoice entry against pre-stored service data and the invoice fee schedule 208, ensuring that the service type, duration, and corresponding fee match contractual and accounting requirements. Similarly, in the approval queue B 514, the internal entity 112 reviews each retrieval invoice entry 504a and helpline invoice entry 504b to confirm that the corresponding customer service tasks were completed, verified, and correctly billed.

During the review process, the at least one processor 104 facilitates retrieval of relevant supporting data for each invoice entry 502a-502c or invoice entry 504a-504b from the invoice management database 114, including timestamps, the service appointment 206 details, and work order 204 identifiers. The computer-readable storage media 106 ensures that the internal entity 112 has secure and role-based access to the approval interface 118 for validating, approving, or rejecting each invoice 404. Upon successful validation, the at least one processor 104 updates the status of the invoice 404 to "approved," marking the individual invoice entries within the invoice 404 as verified. If discrepancies or incomplete data are detected, the at least one processor 104 flags the respective invoice entries and routes the invoice 404 back to the external entity 110 for correction and resubmission through the same approval queue.

In some embodiments, upon approval of the invoice 404, the at least one processor 104 automatically calculates the final payable amount by aggregating validated invoice entries 502a-502c or invoice entries 504a-504b, applying applicable taxes, discounts, or adjustments defined in the invoice fee schedule 208. The approved invoice 404 is then transmitted via the communication network 108 to subsequent administrative modules, such as payment processing or financial reporting systems, ensuring seamless continuity between operational and financial workflows. The computer-readable storage media 106 maintains audit trails of all actionsperformed by the internal entity 112, providing traceability and compliance with regulatory standards.

Accordingly, FIG. 5 depicts the structured approval mechanism of the invoice management system, in which each invoice 404, classified under the invoice type A 502 or the invoice type B 504, is routed to a dedicated approval queue 506 or 514 for verification by the internal entity 112. The described workflow ensures that each invoice entry 502a-502c and invoice entry 504a-504b undergoes systematic validation against operational and financial datasets, thereby improving accuracy, accountability, and transparency of the invoicing process. The coordinated operation of the at least one processor 104, the computer-readable storage media 106, the approval interface 118, and the invoice management database 114 ensures that the invoice management system provides a robust, automated, and auditable mechanism for managing multi- category invoice approvals in real time.

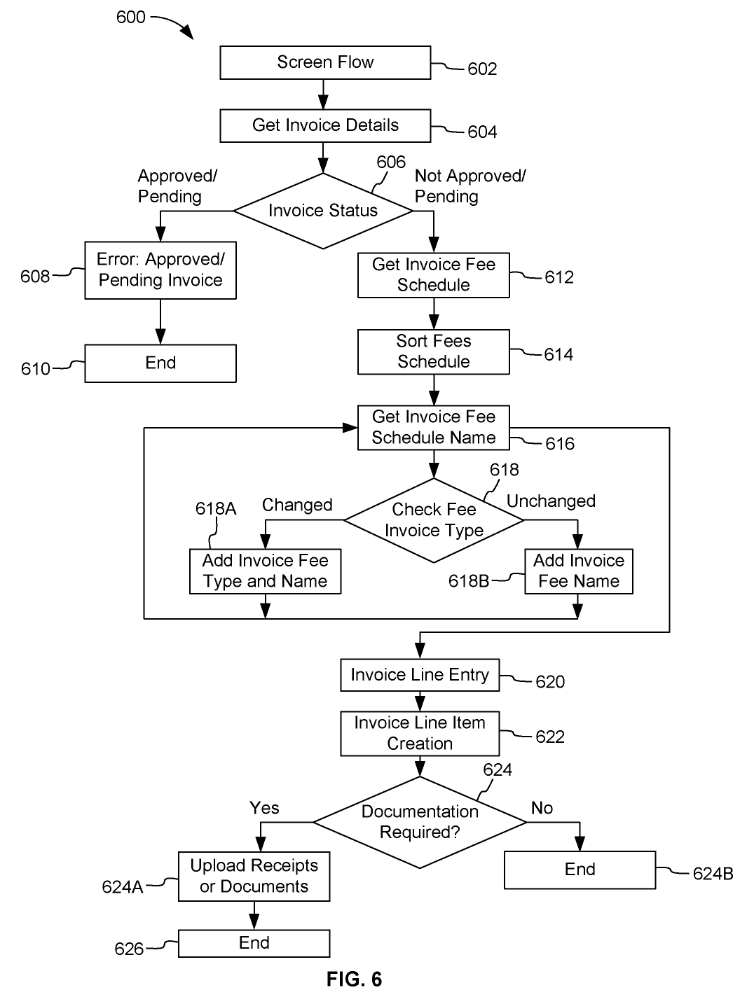

FIG. 6 illustrates a flow chart 600 for a screen flow 602 representing an operational sequence for invoice creation and data entry within the invoice management system, in accordance with one or more embodiments of the present disclosure. FIG. 6 is described in conjunction with FIGS. 1-5. The screen flow 602 defines a logical process through which the at least one processor 104 and the computer-readable storage media 106 cooperate to capture, validate, and associate invoice details and fee schedules during invoice generation. The screen flow 602 enables automated decision-making for determining invoice status, retrieving fee schedules, and associating invoice fee schedules' names prior to the creation of invoice entries 210 and subsequent approval via the approval interface 118.

The screen flow 602 initiates at an operation 604 (Get Invoice Details). At the operation 604, the at least one processor 104 is configured to receive, via the invoice creation interface 116, one or more invoice details corresponding to the invoice 404 created by the external entity 110. The one or more invoice details may include the invoice number, service dates, the invoice type 308, and the description 306. The at least one processor 104 transmits the received invoice details through the communication network 108 to the computer-readable storage media 106, which stores the data within the invoice management database 114 for processing. The at least one processor 104 may be configured to perform real-time validation checks on the received data, including format verification and duplication detection, to ensure data accuracy prior to status evaluation.

Upon completion of the operation 604, the screen flow 602 advances to a decision operation 606 (Invoice Status). At the operation 606, the at least one processor 104 is configured to determine a current invoice status corresponding to the invoice 404. The at least one processor 104 queries the invoice management database 114 to verify whether the invoice 404 has been previously marked as "approved" or "pending." If the invoice status indicates that the invoice 404 is "approved" or "pending," the at least one processor 104 directs the flow 602 to operation 608, which is an Error: Approved/Pending Invoice, signifying that the invoice 404 cannot be modified or reassigned a new fee schedule 208 during this state. Following the error handling at the operation 608, the screen flow 602 terminates at operation 610 (End), thereby concluding the process for the approved or pending invoice without further changes.

When, however, the invoice status 606 is identified as "not approved" or "not pending," the at least one processor 104 proceeds to operation 612 (Get Invoice for Fee Schedule). At the operation 612, the at least one processor 104 is configured to retrieve or receive a corresponding invoice fee schedule 208 for the invoice 404. The at least one processor 104 queries the invoice fee schedule 208 stored in the invoice management database 114 to determine whether a pre-configured fee schedule exists for the invoice 404. When the invoice fee schedule 208 is not pre-configured, the at least one processor 104 provides an interface prompt through the invoice creation interface 116, allowing the external entity 110 to create or select a new invoice fee schedule 208 that aligns with the invoice type 308 and associated service category.

Following the operation 612, the screen flow 602 advances to operation 614 (Sort Fee Schedule). At the operation 614, the at least one processor 104 is configured to sort one or more fee schedule entries retrieved from the invoice fee schedule 208 based on parameters such as service category, task type, fee structure, or GL codes (General Ledger codes). Sorting the invoice fee schedule 208 ensures that the invoice 404 is associated with the relevant fee structure based on the services rendered or the work order 204 linked to the invoice 404. The computer-readable storage media 106 facilitates storage and indexing of the invoice fee schedule 208 in the invoice management database 114 for optimized retrieval and subsequent processing.

At operation 616 which is Get Invoice Fee Schedule Name, the at least one processor 104 is configured to retrieve or generate a corresponding invoice fee schedule name from the invoice management database 114. The invoice fee schedule name serves as a unique reference linking the invoice 404 to its applicable fee structure and task category. The at least one processor104 verifies the invoice fee schedule name for validity and consistency with existing entries to prevent duplication or mismatched linkage. The at least one processor 104 further ensures that the retrieved invoice fee schedule name corresponds to the invoice type 308 (e.g., field service operations 310 or customer service operations 312) selected by the external entity 110, as previously described with reference to FIG. 3.

In some embodiments, upon completing the operation 616, the at least one processor 104 determines (at 618) whether the fee invoice type associated with the invoice 404 has changed relative to the fee invoice type pre-configured in the invoice management database 114. When a change is detected, the at least one processor 104 performs operation 618a (Add Invoice Fee Type and Name), adding both the invoice fee type and the invoice fee schedule name to the invoice 404. The computer-readable storage media 106 processes this association and updates the invoice management database 114 with the new data entries. The at least one processor 104 validates the updated information to confirm alignment between the new invoice fee type, the invoice type 308, and the applicable fee structure within the invoice fee schedule 208.

When the fee invoice type is unchanged, the at least one processor 104 performs operation 618b (Add Invoice Fee Name). At the operation 618b, the at least one processor 104 adds only the invoice fee schedule name to the invoice 404, ensuring that the correct fee schedule name is applied and stored within the invoice management database 114. The computer-readable storage media 106 records the updated linkage between the invoice 404 and the invoice fee schedule 208, maintaining referential integrity across all associated entities. Once the fee-related configurations are finalized, the screen flow 602 proceeds to operation 620, which is Enter Invoice into Invoice Line Entry.

At the operation 620, the at least one processor 104 is configured to enter the invoice 404 into the invoice line entry module, where detailed invoice entries 210 are created. The at least one processor 104 receives, via the invoice creation interface 116, one or more line entry inputs corresponding to individual services rendered or expenses incurred as part of the invoice 404. The computer-readable storage media 106 stores these invoice entries 210 in the invoice management database 114, linking each invoice line entry 210 with the appropriate invoice number 404, the service appointment 206, and work order 204.

The screen flow 602 then advances to operation 622, which is Create Invoice Line Item. At the operation 622, the at least one processor 104 is configured to generate one or more invoice line items corresponding to the invoice entries 210. Each invoice line item may represent an atomic service activity, such as a training service, installation, or retrieval, along with its associated fee structure from the invoice fee schedule 208. The at least one processor 104 validates that each invoice line item is accurately linked to its respective fee amount, GL code, and service date. The computer-readable storage media 106 facilitates the storage and retrieval of these invoice line items for downstream approval and reporting.

Following creation of the invoice line items, the at least one processor 104 performs operation 624, which is Check for Supporting Documentation. At the operation 624, the at least one processor 104 determines whether one or more supporting documents, such as receipts, patient service confirmations, or expense records, are required for the invoice 404. If the determination at the operation 624 indicates that supporting documents are required, the at least one processor 104 executes operation 624a, which is Upload Supporting Documents. At the operation 624a, the external entity 110 is prompted via the invoice creation interface 116 to upload the necessary supporting documents. The computer-readable storage media 106 then stores the uploaded documents in the invoice management database 114, ensuring that each document is linked to the corresponding invoice line entry 210 and invoice 404 for traceability and audit readiness.

In some embodiments, if the determination at the operation 624 indicates that the supporting documents are required, the processor 104 may automatically upload the necessary supporting documents corresponding to the invoice 404 from the invoice management database 114. In a scenario, the user may verify that the automatically uploaded supporting documents are the consistent with the invoice 404.

When, at the operation 624, the at least one processor 104 determines that no supporting documents are required, the process transitions to operation 624b (End Determination of Documentation Requirement). At this operation, the at least one processor 104 finalizes the verification state of the invoice 404 and proceeds to operation 626 (End), effectively concluding the screen flow 602. The completed invoice 404, along with its invoice entries 210, the invoice fee schedule 208, and any supporting documentation, is stored in the invoice management database 114, awaiting review and approval through the approval interface 118 as described with reference to FIG. 5.

Accordingly, FIG. 6 provides a comprehensive view of the stepwise logic implemented by the invoice management system for creating, validating, and configuring invoices prior to approval. The cooperative functionality of the at least one processor 104, the computer- readable storage media 106, the invoice creation interface 116, and the invoice management database 114 ensures that each invoice 404 progresses through a structured validation pipeline. The above-described automated sequence guarantees that only complete, accurately configured invoices proceed to the approval stage, thereby maintaining high data integrity, operational efficiency, and compliance across the entire invoicing workflow.

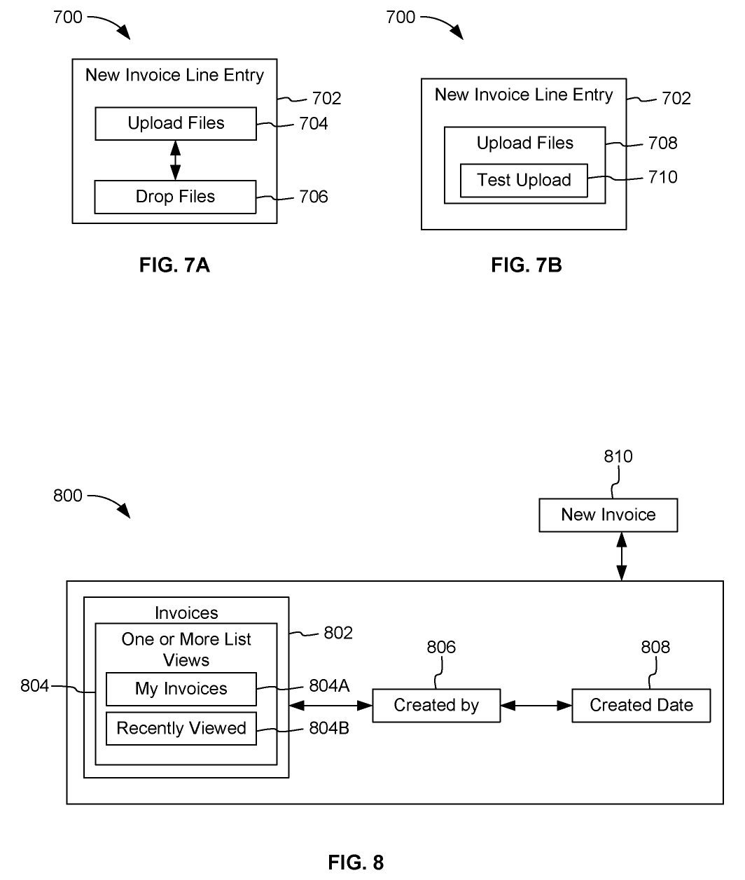

FIGS. 7A and 7B collectively illustrate an interface representation 700 depicting a process for uploading supporting documents corresponding to an invoice line entry within the invoice management system, in accordance with one or more embodiments of the present disclosure. The interface representation 700 is described in conjunction with FIGS. 1-6. As illustrated in FIG. 7A, the interface representation 700 corresponds to a New Invoice Line Entry module through which the external entity 110 may attach one or more supporting documents to a particular invoice 404 or invoice line entry 210. The at least one processor 104 is configured to provide the external entity 110 with multiple upload options via the invoice creation interface 116 to facilitate efficient document submission, ensuring that each uploaded file is automatically linked with its corresponding invoice number and the invoice fee schedule 208 for subsequent validation, review, and approval.

In some embodiments, the interface representation 700 describes a new invoice line entry 702, which includes two upload options, including an Upload Files option 704 and a Drop Files option 706. The Upload Files option 704 may be represented as an interactive button configured to trigger a file explorer window on the external entity's 110 local computing device, allowing selection of one or more supporting documents. The Drop Files option 706 may define a drag-and-drop region within the invoice creation interface 116, into which the external entity 110 directly drops files for upload. The at least one processor 104, in cooperation with the computer- readable storage media 106, manages the initiation of file transfer through the communication network 108 once either of the options 704 or the Drop Files option 706 is used. When the external entity 110 selects the Upload Files option 704, the interface representation 700 transitions to the state illustrated in FIG. 7B. As shown in FIG. 7B, an upload selection interface is displayed, presenting a list of available documents that may be selected for upload. For example, as illustrated, a selectable entry labelled "Test Upload" 710 is presented, representing a document available on the local system of the external entity 110. Upon selection

When the external entity 110 selects the Upload Files option 704, the interface representation 700 transitions to the state illustrated in FIG. 7B. As shown in FIG. 7B, an upload selection interface is displayed, presenting a list of available documents that may be selected for upload. For example, as illustrated, a selectable entry labelled "Test Upload" 710 is presented, representing a document available on the local system of the external entity 110. Upon selectionof the Test Upload 710 option, the at least one processor 104 initiates the file upload process, transferring the selected document to the computer-readable storage media 106 for processing and storage. The computer-readable storage media 106 temporarily caches the uploaded file in the invoice management database 114 or in a designated storage repository while performing integrity and format validation checks. The at least one processor 104 verifies that the uploaded file adheres to predefined system parameters such as file type, file size, and naming convention stored in the computer-readable storage media 106 to ensure compatibility and security compliance during upload.

Once the selected document (for example, the Test Upload 710) is successfully transferred and validated, the interface representation 700 updates dynamically to display an Uploaded Files section 708. Within the Uploaded Files section 708, each uploaded file is listed alongside its corresponding invoice identifier (for example, the invoice number 404 such as "INV- 202501-00699"), confirming successful association with the correct invoice 404 and invoice line entry 210. The at least one processor 104 ensures that each uploaded document is properly indexed in the invoice management database 114 under the respective invoice line entry to enable traceability and auditability. The computer-readable storage media 106 manages metadata generation for each uploaded file, including file name, upload timestamp, uploader identity, and invoice linkage, thereby enabling consistent and efficient document retrieval during subsequent approval queue 506 operations (as described in FIG. 5).

In some embodiments, the invoice management system may be configured to automatically perform document validation and categorization upon upload. The at least one processor 104 may analyze each uploaded document using predefined rules stored in the computer- readable storage media 106 to determine its relevance to specific invoice entries 210, the service appointment 206, or fee categories. For instance, an uploaded training receipt may automatically be classified under training service entries, while a transportation invoice may be categorized under inventory count mileage 404a. Such automatic categorization reduces manual intervention, ensuring that supporting documentation is systematically linked to the appropriate financial and operational record within the invoice management database 114.

During the upload process, the computer-readable storage media 106 encrypts the document data in transit via the communication network 108 and stores it in a secure document repository within the invoice management database 114. The computer-readable storage media106 maintains configuration parameters defining allowed file types (for example, Portable Document Format (PDF), Joint Photographic Experts Group (JPEG), Office Open XML (DOCX)), maximum file size thresholds, and retention policies governing document lifecycle management. The at least one processor 104 monitors compliance with these parameters and may generate system notifications if an upload attempt violates predefined constraints, such as exceeding maximum file size or submitting unsupported formats. Such control ensures consistent adherence to corporate and regulatory compliance standards while maintaining operational efficiency.

In some embodiments, after successful upload, the external entity 110 may be allowed to review or delete previously uploaded documents within the Uploaded Files section 708. The at least one processor 104 provides options within the invoice creation interface 116 for viewing document previews, verifying correctness, or re-uploading corrected versions if required. Any modification made by the external entity 110 is synchronized in real-time with the computer- readable storage media 106, which updates the relevant file metadata and audit trail entries in the invoice management database 114. Each change event, including file additions, deletions, or replacements, is logged by the at least one processor 104 to ensure full traceability and accountability across all uploaded records.

Accordingly, FIGS. 7A and 7B collectively illustrate how the invoice management system facilitates secure, efficient, and traceable document uploads corresponding to invoice line entries/entry 210. The collaborative operation of the at least one processor 104, the computer- readable storage media 106, the invoice creation interface 116, and the invoice management database 114 ensures that each uploaded document is validated, properly linked, and readily accessible for subsequent verification, approval, and audit. This functionality enhances data integrity and ensures that all necessary supporting documents are available for review, thereby improving the reliability and compliance of the overall invoice lifecycle management process.

FIG. 8 illustrates an interface representation 800 depicting an invoice listing and management view within the invoice management system, in accordance with one or more embodiments of the present disclosure.FIG. 8 is described in conjunction with FIGS. 1-7B. The interface representation 800 provides the external entity 110 and the internal entity 112 with a consolidated overview of existing invoices, including options to view, filter, and create new invoices. The interface representation 800 may be rendered within the invoice creation interface 116 and is dynamically generated and controlled by the at least one processor 104 in communication with the computer-readable storage media 106 and the invoice management database 114.

The interface representation 800 includes an invoices section 802, which serves as the primary workspace where users can access invoice-related data stored in the invoice management database 114. The invoices section 802 is organized into one or more list views 804, enabling structured navigation and retrieval of invoice records based on predefined filters or user preferences. Each of the one or more list views 804 provides a categorized display of invoices according to viewing context, facilitating efficient user access and management of invoice data.

In an exemplary embodiment, the one or more list views 804 include my invoices view 804a view and recently viewed view 804b. The my invoices view 804a may be configured to display invoices that are created, owned, or managed by the currently logged-in external entity 110 or internal entity 112. The recently viewed view 804b may display invoices that have been accessed or modified recently within a predefined period. In the illustrated embodiment, the my invoices view 804a is shown as pinned, indicating that this view is prioritized for quick access. The at least one processor 104 maintains these viewing preferences within the computer-readable storage media 106 to enable a personalized and consistent user experience across sessions.

The interface representation 800 further includes a set of informational columns displayed adjacent to the list views 804. These include Created By column 806 and Created Date column 808. The Created By column 806 identifies the external entity 110 or internal entity 112 responsible for generating or submitting a particular invoice within the invoice management database 114, while the Created Date column 808 displays the timestamp indicating when the invoice record was initially created. The at least one processor 104 retrieves these details from the invoice management database 114 in real-time to ensure that all displayed information accurately reflects the current system data.

In some embodiments, the interface representation 800 also includes new invoice option 810 located in the upper-right portion of the interface. The new invoice option 810 serves as an interactive control that enables the external entity 110 or internal entity 112 to initiate the invoice creation process directly from the invoices section 802. Upon activation of the new invoice option 810, the at least one processor 104 transitions the invoice creation interface 116 to the new invoice screen as described in FIG. 3, allowing entry of details such as the invoice type 308, status 304, and the description 306. This seamless transition ensures that the workflow from invoice listing to invoice creation remains efficient and consistent within the invoice management system.

The computer-readable storage media 106 may be configured to synchronize the data presented within the interface representation 800 with updates occurring elsewhere in the invoice management system. For example, when a new invoice is created through the new invoice option 810, approved via an approval queue 506 (as described in FIG. 5), or modified through the invoice line entry 210 process (as described in FIG. 4), the computer-readable storage media 106 automatically updates the relevant records in the invoice management database 114. The at least one processor 104 then refreshes the invoices section 802 to display the up-to-date invoice statuses, creation details, and approval outcomes.

Additionally, the interface representation 800 may support filtering, sorting, and search functionalities implemented through the invoice creation interface 116. The at least one processor 104 may be configured to apply user-defined filters on parameters such as invoice status, creation date, or creator identity, enabling the external entity 110 to quickly locate specific invoices. The computer-readable storage media 106 may store user preferences related to these filters and sorting options to ensure a consistent and customized experience across multiple user sessions.

Through these components, the interface representation 800 serves as a centralized access point for managing the lifecycle of invoices within the invoice management system. The coordinated functioning of the at least one processor 104, the computer-readable storage media 106, the invoice management database 114, and the invoice creation interface 116 ensures that the external entity 110 and the internal entity 112 can efficiently view, create, and manage invoice records. The interface thereby enhances transparency, improves navigability, and ensures that all invoicing activities within the computer environment 100 are recorded, retrievable, and up-to-date in real time.

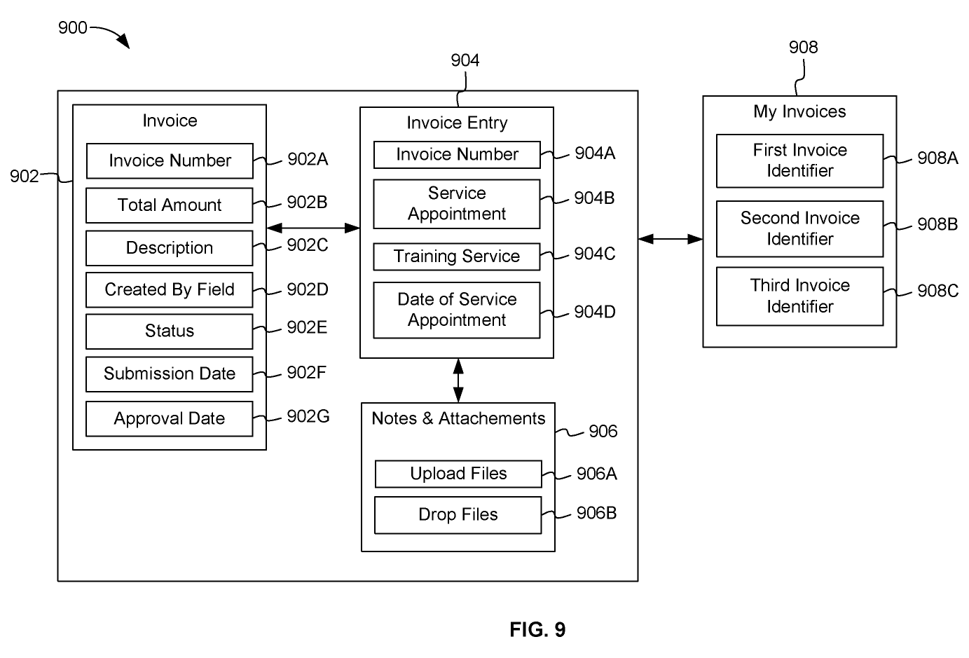

FIG. 9 illustrates an interface representation 900 depicting an integrated view of an invoice 902, an invoice entry 904, and associated notes and attachments 906 within the invoice management system, in accordance with one or more embodiments of the present disclosure. FIG. 9 is described in conjunction with FIGS. 1-8. The interface representation 900 provides a comprehensive visualization of how invoice data, related entries, and supporting documents are displayed and managed within the invoice creation interface 116. The interface representation 900 also demonstrates the multi-device accessibility of the invoice management system, wherein the same data representation may be simultaneously displayed on both a laptop-based interface and a mobile-based interface. The at least one processor 104 may be configured to render, synchronize, and manage these displays by interacting with the computer-readable storage media 106 and the invoice management database 114 over the communication network 108.

The invoice 902 presents core information corresponding to an invoice record stored in the invoice management database 114. The invoice 902 includes an invoice number 902a, a total amount 902b, a description 902c, a created-by field 902d, a status 902e, a submission date 902f, and an approval date 902g. Each of these fields corresponds to data attributes within the invoice table associated with the invoice management database 114. The at least one processor 104 retrieves and displays these data attributes in real time whenever the external entity 110 or the internal entity 112 accesses a specific invoice through the invoice creation interface 116. For example, the invoice number 902a uniquely identifies the invoice; the total amount 902b reflects the cumulative value of all invoice entries 210 linked to that invoice; and the status 902e represents whether the invoice is in draft, submitted, approved, or rejected state as maintained within the invoice management database 114.

The created-by field 902d identifies the external entity 110 or internal entity 112 responsible for generating the invoice 902, while the submission date 902f and the approval date 902g provide temporal references that support audit tracking and reporting. The at least one processor 104 may be configured to automatically populate the submission date 902f upon submission of the invoice 902 and update the approval date 902g when the invoice is approved through an approval queue 506 (as described in FIG. 5). These updates may be propagated throughout the invoice management system to ensure real-time visibility of invoice progress for both the external entity 110 and the internal entity 112.

Adjacent to the invoice 902, the interface representation 900 includes an invoice entry 904 that lists the detailed components making up the invoice 902. The invoice entry 904 may include fields such as invoice number 904a, service appointment 904b, training services 904c, and date of service appointment 904d. Each of these fields corresponds to data stored within the invoice line entry 210 as described in FIG. 2 and FIG. 4. The at least one processor 104 may be configured to link each field of the invoice 904 to its corresponding invoice record by referencing relational mappings within the invoice management database 114. For instance, when the external entity 110 selects a specific invoice entry 904 from the invoice entry 904 section, the invoice creation interface 116 may expand the entry to reveal associated details such as training type, fee structure (as obtained from the invoice fee schedule 208), and related service appointment information.

Below the invoice entry 904 section, the interface representation 900 includes notes and attachments 906 that facilitates document association with the invoice 902. The notes and attachments 906 may provide the external entity 110 with options such as an Upload Files 906a option and a Drop Files 906b option allowing the external entity 110 to attach receipts, field reports, or supporting documentation corresponding to the invoice 902. The at least one processor 104 may be configured to process these uploads through the computer-readable storage media 106 and store them in the invoice management database 114 or an associated document repository, while maintaining logical links to the correct invoice 902 and invoice entry 904. Once the files are successfully uploaded, the invoice creation interface 116 displays the uploaded file identifiers and metadata within the notes and attachments 906, confirming successful association of the documents with the invoice record.

In some embodiments, the interface representation 900 also demonstrates cross- platform accessibility by including a mobile interface display labelled My Invoices 908, which provides a condensed view of invoices accessible to the external entity 110. The My Invoices 908 displays one or more invoices represented by invoice identifiers, such as a first invoice identifier 908a (INV-202408-00006), a second invoice identifier 908b (INV-202408-00007), and a third 908c (INV-202408-00008). Each invoice identifier in the My Invoices 908 view corresponds to the invoice 902 stored in the invoice management database 114 and may be selected to access detailed invoice information similar to that displayed on the laptop-based interface. The at least one processor 104 ensures that both interfaces remain synchronized in real time through the communication network 108, enabling continuity of operations across devices.

In an exemplary embodiment, when the external entity 110 or internal entity 112 updates the invoice entry 904 or uploads a document via the notes and attachments 906 on one device, the computer-readable storage media 106 propagates the corresponding changes to all active sessions. As a result, the My Invoices 908 on the mobile interface immediately reflects the updated invoice status 902e or newly added attachments. This synchronization ensures data consistency across multiple device types and user interfaces within the invoice management system. The computer-readable storage media 106 maintains the underlying synchronization logicand user session data, while the at least one processor 104 executes data rendering and update propagation in real time.

Accordingly, the interface representation 900 collectively demonstrates how the invoice management system provides comprehensive, device-independent management of invoice details, associated entries, and supporting documentation. By enabling both detailed laptop-based data entry and summarized mobile access through the invoice creation interface 116, the system ensures that the external entity 110 and the internal entity 112 can monitor, edit, and verify invoice- related information efficiently and accurately. The combined operation of the at least one processor 104, the computer-readable storage media 106, the invoice management database 114, and the communication network 108 guarantees real-time visibility, integrity, and traceability of all invoice-related activities within the computer environment 100.

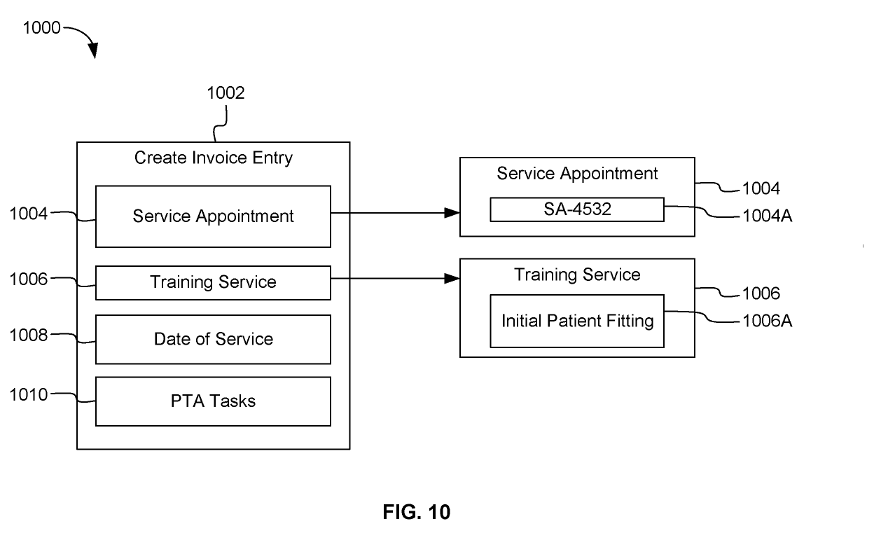

FIG. 10 illustrates an interface representation 1000 depicting a user interface for creating a new invoice line entry within the invoice creation interface 116 of the invoice management system, in accordance with one or more embodiments of the present disclosure. FIG. 10 is described in conjunction with FIGS. 1-9. The interface representation 1000 enables the external entity 110 to enter detailed service and task information corresponding to a specific invoice 202 being created or modified. The interface representation 1000 is configured to guide the external entity 110 through the entry of required data fields that collectively constitute an invoice line entry 210, ensuring accuracy, uniformity, and traceability across all invoices processed within the invoice management database 114.

The interface representation 1000 includes a create invoice entry section 1002, which serves as the primary workspace for entering or editing invoice line details. The create invoice entry section 1002 may be dynamically generated by the at least one processor 104 when the external entity 110 selects the option to add a new invoice line item under the invoice creation interface 116. The create invoice entry section 1002 displays contextual input fields corresponding to relevant service, scheduling, and task information associated with a specific invoice line entry 210. Each input field is semantically linked to data attributes maintained within the invoice management database 114 and validated by the computer-readable storage media 106 before storage.

As illustrated, the create invoice entry section 1002 includes a service appointment 1004, a training service 1006, a date of service 1008, and PTA tasks 1010. The service appointment 1004 represents the specific operational or service instance to which the invoice line pertains. The service appointment 1004 may include a unique identifier such as SA-4532, shown as service appointment 1004a, which corresponds to a distinct record in the work order 204 or service appointment table maintained within the invoice management database 114. The at least one processor 104 may be configured to retrieve available service appointment identifiers dynamically based on assigned service portfolio of the external entity 110, ensuring that only valid and authorized service appointments are selectable within the create invoice entry section1002.

The training service 1006 corresponds to the nature of service performed during the service appointment 1004 and may include options such as "Initial Patient Fitting 1006a," represented under training service 1006 in FIG. 10. The training service 1006 is associated with the invoice fee schedule 208 described in FIG. 2, which defines the corresponding fee type, billing rate, and service category applicable to the selected training service 1006. The at least one processor 104 may be configured to automatically populate or validate the training service 1006 by referencing the invoice fee schedule 208 to ensure that the correct fee type is applied to the invoice line entry 210. This linkage ensures pricing integrity, compliance with billing rules, and consistency in financial reporting across all invoices generated within the invoice management system.

The date of service 1008 captures the actual date on which the training service 1006 or service appointment 1004 occurred. In the illustrated example, the date of service 1008 displays a calendar interface where a date such as 2 July 2024 is selected. The at least one processor 104 may be configured to provide an interactive date selector allowing the external entity 110 to select a valid service date that corresponds with the service appointment 1004. The at least one processor 104 validates the selected date against system records, ensuring that the date of service 1008 does not precede the creation date of the service appointment 1004 or fall outside permitted reporting intervals. Once validated, the date of service 1008 is stored within the invoice line entry 210 record in the invoice management database 114, enabling chronological tracking and audit compliance.

The PTA tasks 1010 represent the specific activities performed under the training service 1006 as part of the Patient Training Agreement (PTA). The PTA tasks 1010 may include predefined categories such as device setup, calibration, patient orientation, or follow-up support. The at least one processor 104 may be configured to allow the external entity 110 to select or input relevant PTA tasks 1010 associated with the service appointment 1004. The at least one processor104 cross-references these tasks with the applicable invoice fee schedule 208 and ensures that the selected tasks are compliant with contractual billing parameters associated with the external entity 110. Each task entered under the PTA tasks 1010 is stored as part of the invoice line entry 210 and linked to the corresponding training service 1006 and service appointment 1004 to maintain full traceability.

In some embodiments, the interface representation 1000 may further display validation prompts, automated checks, or contextual suggestions to assist the external entity 110 in ensuring completeness and accuracy of the entered data. The at least one processor 104 may be configured to trigger real-time validation of the service appointment 1004, training service 1006, date of service 1008, and PTA tasks 1010 before the invoice line entry 210 is finalized. When discrepancies or missing values are detected, the at least one processor 104 generates an error notification prompting corrective action by the external entity 110. Once all fields are successfully validated, the invoice line entry 210 is stored in the invoice management database 114 and made available for further processing or approval, as illustrated in FIG. 5.

Accordingly, the interface representation 1000 provides a structured and guided environment for the external entity 110 to accurately create and manage new invoice line items within the invoice management system. By linking the service appointment 1004, training service 1006, date of service 1008, and PTA tasks 1010 directly to existing data models such as the invoice fee schedule 208 and the work order 204, the system ensures consistency, transparency, and auditability across all invoice records. The combined operation of the at least one processor 104, the computer-readable storage media 106, the invoice management database 114, and the communication network 108 enables real-time validation, data synchronization, and secure storage of all invoice entries created through the invoice creation interface 116, thereby ensuring high data fidelity and compliance within the computer environment 100.

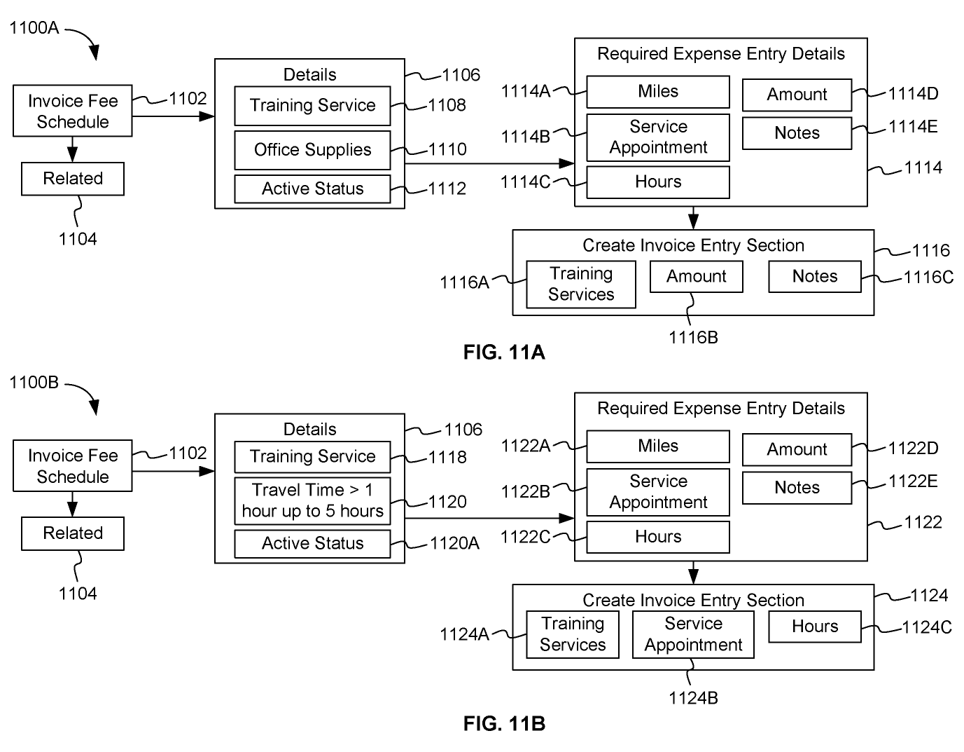

FIGS. 11A and 11B illustrate interface representations 1100A and 1100B, respectively, depicting examples of invoice fee schedule configurations and corresponding invoice entry creation interfaces within the invoice management system, in accordance with one or more embodiments of the present disclosure. FIGS. 1lA and 11B are described in conjunction with FIGS. 1-10. The interface representations 1100A and 1100B collectively demonstrate how the external entity 110 selects, configures, and applies an invoice fee schedule 208 to generate a corresponding invoice line entry 210 through the invoice creation interface 116. Each of the interface representations 1100A and 1100B is rendered by the at least one processor 104 to facilitate seamless association of a specific training service or expense category with its predefined billing parameters and related data fields stored within the invoice management database 114.

As illustrated in FIG. 11A, the interface representation 1100A includes an invoice fee schedule 1102 corresponding to a training service categorized as "Office Supplies." The invoice fee schedule 1102 represents a predefined structure within the invoice management database 114 that stores fee rates, expense configurations, and task associations for specific service types. The invoice fee schedule 1102 includes two selectable tabs, namely a related tab 1104 and a details tab 1106. The details tab 1106 is shown as being selected by the external entity 110 in FIG. 11A. Upon selection, the details tab 1106 expands to reveal a set of attributes that define the configuration of the invoice fee schedule 1102, including training service 1108, office supplies 1110, and active status 1112.

The training service 1108 defines the category of service associated with the invoice fee schedule 1102, linking the "Office Supplies" expense type to its relevant training or support activities. The office supplies 1110 specifies the particular cost center or service context under which the invoice fee schedule applies. The active status 1112 indicates that the fee schedule is currently valid and available for use in invoice creation. In some embodiments, the at least one processor 104 may be configured to validate that the active status 1112 is true before enabling the creation of a new invoice entry. This validation ensures that outdated or inactive fee schedules are not utilized during invoice generation, maintaining billing accuracy and compliance with contractual pricing rules.

Under the details 1106 tab, the invoice fee schedule 1102 further includes required expense entry details 1114, which define specific expense parameters associated with the training service 1108. The required expense entry details 1114 include data fields such as miles 1114a, service appointment 1114b, hours 1114c, amount 1114d, and notes 1114e. Each of these fields represents a configurable expense or data element required for constructing the invoice line entry 210. For example, the miles 1114a allows the external entity 110 to record travel-related distances when applicable; the service appointment 1114b links the expense to a specific service appointment 1004 (as described in FIG. 10); the hours 1114c specifies the time spent on the corresponding service; the amount 1114d captures the cost associated with the recorded expense; and the notes 1114e provides a space for contextual annotations.

Upon completion of the required expense entry details 1114, the interface representation 1100A proceeds to a create invoice entry section 1116. The create invoice entry section 1116 represents the stage where the external entity 110 generates a new invoice line entry 210 linked to the invoice fee schedule 1102. The create invoice entry section 1116 includes structured data such as training services 1116a, amount 1116b, and notes 1116c. In the illustrated embodiment, the training services 1116a reflects the category "Office Supplies," which is inherited from the training service 1116a. The at least one processor 104 may be configured to automatically populate the amount 1116b using fee values defined within the invoice fee schedule 208, thereby reducing manual entry and preventing billing inconsistencies. The notes 1116c allows the external entity 110 to include supplementary information relevant to the created invoice line entry 210.

As illustrated in FIG. 11B, the interface representation 1100B demonstrates another configuration of the invoice fee schedule 208, corresponding to a training service titled "Travel Time more than 1 hour up to 5 hours." Similar to FIG. 11A, the invoice fee schedule 1102 in FIG. 11B is presented with the related tab 1104 and the details tabs 1106, with the details tab 1106 expanded to reveal applicable parameters. The details tab 1106 includes a training service 1118, which in this case corresponds to a travel time 1120, for example, "Travel Time more than 1 hour up to 5 hours," and an active status 1120a. This configuration allows the invoice management system to associate travel-related time segments with corresponding fee rates, ensuring accurate and rule-based invoice computation.

The invoice fee schedule 1102 in FIG. 11B further defines required expense entry details 1122, which includes miles 1122a, service appointment 1122b, hours 1122c, amount 1122d, and notes 1122e. The at least one processor 104 may be configured to enforce input validation rules for each of these fields to ensure that recorded travel hours or miles correspond with permissible billing limits defined within the invoice fee schedule 208. For instance, the hours 1122c may be limited to a numerical range consistent with the "more than 1 hour up to 5 hours" category.

Upon entry of the required details, the interface representation 1100B proceeds to a create invoice entry section 1124, which allows the external entity 110 to finalize and store the newly created invoice line entry 210 associated with the selected training service 1108. The create invoice entry section 1124 includes the fields training service 1124a, service appointments 1124b, and hours 1124c. The training service 1124a reflects "Travel Time more than 1 hour up to 5 hours" as configured in the invoice fee schedule 1102, while the service appointments 1124b links the entry to a corresponding service appointment 1004, ensuring relational consistency within the invoice management database 114. The hours 1124c captures the time value entered by the external entity 110, which is automatically validated and priced according to the applicable rate stored in the invoice fee schedule 208.

Accordingly, FIGS. 11A and 11B collectively illustrate the structured process of linking invoice fee schedules to invoice entries within the invoice management system. The at least one processor 104, in coordination with the computer-readable storage media 106 and the invoice management database 114, ensures that all invoice entries created through the invoice creation interface 116 adhere to pre-defined billing structures, validation rules, and service-specific fee parameters. This configuration guarantees accuracy, consistency, and compliance during invoice creation while providing the external entity 110 with a streamlined and intuitive interface for managing expense-based service records and generating traceable invoice data.

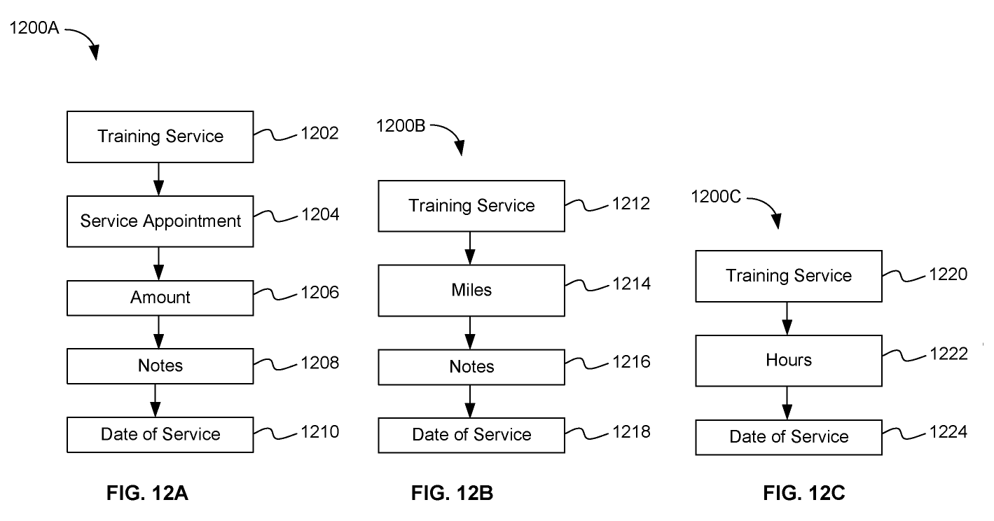

FIGS. 12A, 12B, and 12C illustrate interface representations 1200A, 1200B, and 1200C, respectively, each depicting examples of training service records and their associated data fields as displayed within the invoice creation interface 116 of the invoice management system, in accordance with one or more embodiments of the present disclosure. FIGS. 12A-12C are described in conjunction with FIGS. 1-11. Each of the interface representations 1200A, 1200B, and 1200C demonstrate how the external entity 110 may input or review specific training service details corresponding to individual invoice entries, where each training service is linked to a predefined invoice fee schedule 208 and related operational data such as service appointments 1004, dates of service 1008, and notes captured during the provision of the service.

As illustrated in FIG. 12A, the interface representation 1200A includes a training service 1202 identified as "Parking." The training service 1202 corresponds to a predefined task type or expense category under the applicable invoice fee schedule 208. The interface representation 1200A further includes a service appointment 1204, such as "SA-2610," which identifies the specific service event associated with the training service 1202, which may further have an amount 1206. Notes 1208 records supplementary information relevant to the training service 1202, in this case "Parking for fitting," which describes the nature of the expense in relation to the corresponding service appointment 1204. Date of service 1210 captures the date on which the training service 1202 was performed, such as "July 02, 2024." The at least one processor 104may be configured to validate that the date of service 1210 corresponds with the service appointment 1204 and that the training service 1202 is a permissible expense type defined within the invoice fee schedule 208.

FIG. 12B illustrates another interface representation 1200B, which corresponds to a training service 1212 categorized as "Mileage." The training service 1212 represents travel-related activities performed by the external entity 110 in connection with an assigned service appointment 1004. The interface representation 1200B includes miles 1214 displaying the travel distance entered by the external entity 110, such as "15.00" miles, which may be used to automatically calculate an expense fee based on per-mile rates defined within the invoice fee schedule 208. Notes 1216 records descriptive information for traceability and verification, such as "Roundtrip for SA- 4532." Date of service 1218 records the service date as "July 02, 2024," ensuring that the recorded mileage is properly associated with the corresponding service event. The at least one processor 104 may be configured to automatically retrieve the applicable mileage rate from the invoice fee schedule 208, compute the corresponding cost, and update the invoice line entry 210 stored within the invoice management database 114.

FIG. 12C illustrates the interface representation 1200C, which corresponds to a training service 1220 identified as "Service Cells." The training service 1220 represents a time- based operational activity logged by the external entity 110 as part of servicing a wearable medical device or performing a related task. The interface representation 1200C includes hours 1222, which records the total time spent performing the training service 1218, such as "10 hours". Date of service 1224 records the operational date, such as "July 02, 2024." In some embodiments, the at least one processor 104 may be configured to validate that the hours 1222 conforms to acceptable limits defined in the invoice fee schedule 208 and to calculate the corresponding fee based on the hourly rate structure linked to the training service 1220. The resulting fee and service details may then be associated with the relevant invoice 202 and stored in the invoice management database 114 for approval and further processing through the invoice approval interface 118.

Accordingly, FIGS. 12A-12C collectively illustrate the mechanism by which individual training services such as "Parking," "Mileage," and "Service Cells" are recorded, validated, and linked to corresponding the service appointments 1004 and invoice fee schedule 208 within the invoice management system. Each interface representation 1200A, 1200B, and 1200C provides structured data fields enabling the external entity 110 to document all relevantservice details, thereby allowing the at least one processor 104 to dynamically compute associated fees, ensure data consistency, and maintain traceable records for subsequent approval, reporting, or payment processing. This integrated workflow ensures that all invoice entries reflect accurate, auditable, and system-verified service information aligned with predefined operational and billing parameters.

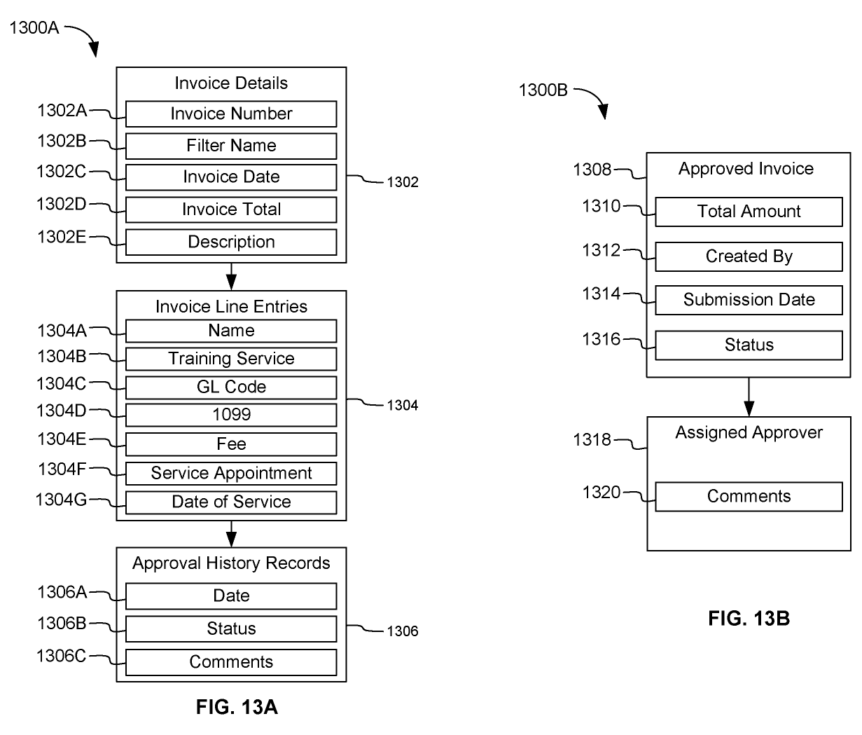

FIGS. 13A and 13B collectively illustrate interface representations 1300A and 1300B, respectively, each showing examples of finalized and approved invoices within the invoice management system, in accordance with one or more embodiments of the present disclosure. FIGS. 13A-13B are described in conjunction with FIGS. 1-12. The interface representations 1300A and 1300B enable both the external entity 110 and the internal entity 112 to visualize, verify, and manage invoice details once all invoice line entries have been created, validated, and processed through the invoice approval interface 118. Each interface representation consolidates comprehensive invoice metadata, corresponding service line details, and approval records, allowing seamless traceability and auditing across the invoice lifecycle.

As illustrated in FIG. 13A, the interface representation 1300A includes invoice details 1302, which encapsulate primary information related to a specific invoice 202. The invoice details 1302 may include an invoice number 1302a, uniquely identifying the invoice within the invoice management database 114, a filter name 1302b, which allows the user to categorize or search invoices by predefined attributes or associated service types, an invoice date 1302c, indicating the date on which the invoice was created or submitted, an invoice total 1302d, which represents the cumulative financial value derived from all included invoice line entries 210, and a description 1302e, which provides a summary or contextual information about the purpose or contents of the invoice.

The at least one processor 104 may be configured to automatically populate these fields based on data previously entered through the invoice creation interface 116 and validated via the integration module 122. The at least one processor 104 may retrieve and present this information dynamically, ensuring the external entity 110 and internal entity 112 view consistent and accurate records reflective of system-verified financial computations.

Beneath the invoice details 1302, the interface representation 1300A further includes invoice line entries 1304, which detail each individual transaction or service item contributing to the invoice total 1302d. The invoice line entries 1304 may include a name 1304a, representing theline entry title or associated service; training services 1304b, denoting the specific task or expense category (for example, "Mileage," "Parking," or "Service Cells," as described in FIGS. 12A-12C); a GL code 1304c, corresponding to the general ledger classification used for accounting and expense tracking within organizational financial systems; and "1099"status 1304d, which indicates whether the payment associated with the line entry is subject to the U.S. Internal Revenue Service Form 1099 reporting requirements, typically applicable for independent contractors or non- employee payments. Fee 1304e specifies the calculated charge for the corresponding service, derived from the invoice fee schedule 208. Service appointment 1304f identifies the relevant appointment or operational event linked to the service, while date of service 1304g records the actual date on which the service was provided.

In some embodiments, the at least one processor 104 may be configured to validate that each invoice line entry 1304 corresponds to a legitimate service appointment 1004 and that the fee 1304e aligns with approved rates stored in the invoice fee schedule 208. The at least one processor 104 may ensure that these details are properly synchronized across user interfaces and stored securely within the invoice management database 114 for subsequent retrieval, reporting, or audit purposes.

Following the invoice line entries 1304, the interface representation 1300A further includes approval history records 1306, which capture the chronological sequence of approval- related activities associated with the invoice 202. The approval history records 1306 may include a date 1306a, representing the timestamp of each approval or review action; a status 1306b, which may indicate "Submitted," "Approved," "Rejected," or "Pending Review"; and comments 1306c, enabling authorized reviewers or approvers (for example, from the internal entity 112) to document justifications, feedback, or clarifications associated with each review action.

In some embodiments, the at least one processor 104 may be configured to automatically update the approval history records 1306 whenever an action is performed through the invoice approval interface 118. The at least one processor 104 may manage version control, ensuring that each approval event is time-stamped, user-identified, and preserved within the invoice management database 114 for compliance and auditing purposes.

As illustrated in FIG. 13B, the interface representation 1300B corresponds to an approved invoice view, providing a summarized and finalized visualization of a validated invoice 1308 once it has been reviewed and authorized by the internal entity 112. The invoice 1308 maydisplay an overall total amount 1310, which represents the cumulative value of all approved line entries. Additionally, created by section 1312 identifies the external entity 110 or user responsible for generating the invoice, while submission date 1314 records the date the invoice was initially submitted for review. Status 1316 displays the current approval state of the invoice (for example, "Approved," "Pending," or "Returned for Revision"). An assigned approver 1318 identifies the authorized personnel or team within the internal entity 112 responsible for the approval action, while comments 1320 provides an area for approvers to record notes or feedback corresponding to the approval decision.

In some embodiments, the at least one processor 104 may be configured to automatically update the status 1316 and assigned approver 1318 in real-time as approval actions are executed within the invoice approval interface 118. The at least one processor 104 may facilitate secure data exchange between the user interfaces and the invoice management database 114, ensuring that the external entity 110 receives immediate feedback on invoice approval outcomes. Additionally, the notification module 124 may be triggered upon approval, rejection, or reassignment, thereby notifying relevant stakeholders through preconfigured communication channels.

The invoice management system thus provides a comprehensive mechanism for the external entity 110 and internal entity 112 to manage the full lifecycle of invoices, from creation and validation to review, approval, and record-keeping. By consolidating invoice details 1302, invoice line entries 1304, and approval history records 1306 (FIG. 13A) with the approved invoice summary (FIG. 13B), the system ensures data consistency, transparency, and compliance across operational, financial, and administrative workflows.

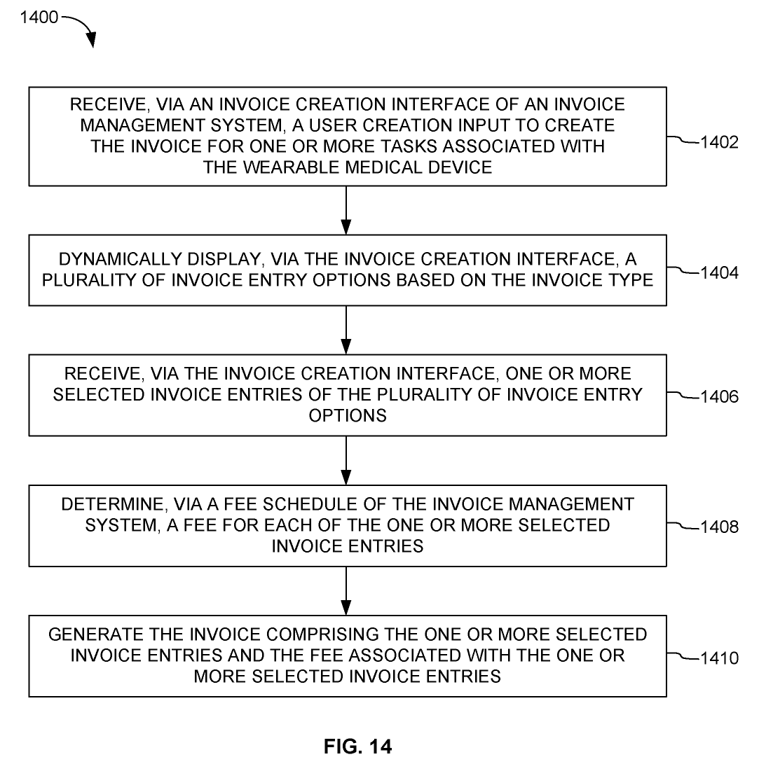

Referring now to FIG. 14, illustrated is a method 1400 for generating an invoice for one or more tasks associated with a wearable medical device, in accordance with an embodiment of the present disclosure. The method 1400 may be implemented by the invoice management system, including execution by the at least one processor 104 in conjunction with program instructions stored on the computer-readable storage media 106. One or more software modules, including the invoice creation interface 116, the fee schedule 208, and the invoice generation module, may be invoked in a coordinated manner to conduct the operations described in the method 1400. FIG. 14 is explained in conjunction with the description of previous figures.

The method 1400 begins at step 1402, where the at least one processor 104 receives, via the invoice creation interface 116 of the invoice management system, a user creation input to create the invoice for one or more tasks associated with the wearable medical device. The user creation input may include an invoice type, which identifies a predefined category of service or expense, such as training services, travel, or office-related costs. The invoice creation interface 116 may validate the received input to ensure completeness, consistency, and adherence to organizational rules before proceeding.

At step 1404, the at least one processor 104 dynamically displays, via the invoice creation interface 116, a plurality of invoice entry options based on the invoice type. Each of the plurality of invoice entry options corresponds to a specific task, expense, or service that is relevant to the selected invoice type. For example, if the invoice type corresponds to "Training Services," the displayed options may include parking, mileage, service hours, or other related expenses, as illustrated in FIGS. 12A-12C. The invoice creation interface 116 may filter and arrange the options in real-time based on previous selections, historical entries, or applicable service schedules, providing the user with an intuitive and contextualized selection experience.

At step 1406, the at least one processor 104 receives, via the invoice creation interface 116, one or more selected invoice entries from the plurality of displayed options. The user may select multiple entries corresponding to different tasks or expenses, for example, a combination of service hours, mileage, and parking for a particular service appointment. The invoice creation interface 116 validates the selections for consistency, ensuring that no duplicate or conflicting entries are included and that all mandatory fields associated with each selection are completed.

At step 1408, the at least one processor 104 determines, via a fee schedule 208 of the invoice management system, a fee for each of the one or more selected invoice entries. The fee schedule 208 may store preconfigured rates for each type of task or expense, including standard rates for training services, mileage reimbursement, and office-related expenditures. The at least one processor 104 may retrieve the applicable rate, apply any user-specific or entity-specific adjustments, and calculate the fee for each selected invoice entry. In some embodiments, the fee calculation may also incorporate conditional rules, such as time-based rates, travel distance thresholds, or service-specific multipliers.

At step 1410, the at least one processor 104 generates the invoice 202 comprising the one or more selected invoice entries and the fees associated with each entry. The invoice generation module consolidates the entries, calculates the total amount, and organizes the information into a structured invoice format, consistent with the interface representations described in FIGS. 8-13B. The generated invoice 202 may include line items, service appointment references, dates of service, notes, and total amounts. In some embodiments, the at least one processor 104 may store the generated invoice in the invoice management database 114, update related approval workflows, and provide options for submission, review, or export.

In summary, the method 1400 enables a systematic, user-guided, and automated approach for generating invoices for tasks associated with wearable medical devices. By dynamically presenting relevant invoice entry options, validating user selections, computing fees from a preconfigured fee schedule, and consolidating the entries into a finalized invoice, the method 1400 ensures accuracy, efficiency, and traceability across the invoice lifecycle. The operations described in method 1400 are consistent with and supported by the hardware and software components detailed in FIGS. 1-13B, including the invoice creation interface 116, the fee schedule 208, and the invoice generation module.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

Other embodiments include combinations and sub-combinations of features described or shown in the drawings herein, including for example, embodiments that are equivalent to: providing or applying a feature in a different order than in a described embodiment, extracting an individual feature from one embodiment, and inserting such feature into another embodiment; removing one or more features from an embodiment; or both removing one or more features from an embodiment and adding one or more features extracted from one or more other embodiments while providing the advantages of the features incorporated in such combinations and sub- combinations. As used in this paragraph, feature or features can refer to the structures and/or functions of an apparatus, article of manufacture or system, and/or the steps, acts, or modalities of a method.

These and other changes can be made to the embodiments in light of the above- detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

In construing the claims of this document, the inventor(s) invoke 35 U.S.C. § 112(f) only when the words "means for" or "steps for" are expressly used in the claims. Accordingly, if these words are not used in a claim, then that claim is not intended to be construed by the inventor(s) in accordance with 35 U.S.C. § 112(f).

What is claimed is:

1. A method for generating an invoice related to servicing a wearable medical device for a patient, the method comprising:
receiving, via an invoice creation interface of an invoice management system, a user creation input to create the invoice for one or more tasks associated with the wearable medical device, the user creation input comprising an invoice type;
dynamically displaying, via the invoice creation interface, a plurality of invoice entry options based on the invoice type, wherein the plurality of invoice entry options are associated with the one or more tasks and/or one or more expenses;
receiving, via the invoice creation interface, one or more selected invoice entries of the plurality of invoice entry options;
determining, via a fee schedule of the invoice management system, a fee for each of the one or more selected invoice entries; and
generating the invoice comprising the one or more selected invoice entries and the fee associated with the one or more selected invoice entries.

2. The method of claim 1, further comprising:
subsequent to receiving the one or more selected invoice entries, dynamically displaying, via the invoice creation interface, a plurality of subfields, wherein the plurality of subfields displayed depend on the plurality of invoice entry options such that one invoice entry option has different subfields from another invoice entry option; and
receiving, via the invoice creation interface, one or more user subfield inputs into the plurality of subfields, thereby providing information related to each of the one or more selected invoice entries.

3. The method of claim 2, wherein the information includes a description of the one or more tasks performed or information related to the one or more expenses.

4. The method of claim 1, the method further comprising:
adding and/or removing, via an administrator interface of the invoice management system, invoice entries options of the plurality of invoice entry options; and/or
adding and/or removing one or more subfields for at least one invoice entry option of the plurality of invoice entry options.

5. The method of claim 1, further comprising:
prior to receiving the user creation input, receiving, at a contractor computing device, a request to perform the one or more tasks related to the wearable medical device;
accepting, via the contractor computing device, the request, thereby generating a work order, wherein the work order is associated with the invoice;
providing the patient with the wearable medical device; and
performing the one or more tasks associated with the wearable medical device.

6. The method of claim 5, the method further comprising automatically adding one or more invoice entries to the invoice based on the work order and/or automatically populating one or more invoice entries based on the work order.

7. The method of claim 5, the method further comprising:
storing, via the invoice management system, at least one of the work order or one or more additional supporting documents in a database;
prior to generating the invoice, detecting, via the invoice management system, an inconsistency in the one or more selected invoice entries based on at least one of the work order or the one or more additional supporting documents; and
transmitting, via the invoice creation interface, one or more requests to update the one or more selected invoice entries based on the inconsistency.

8. The method of claim 1, the method further comprising approving, via an invoice approval interface of the invoice management system, the invoice.

9. The method of claim 8, the method further comprising automatically sending, via the invoice management system, a status communication to a user when a status of the invoice has changed, wherein the status includes submitted, approved, or rejected.

10. The method of claim 9, the method further comprising upon approval of the invoice, generating a form including the one or more selected invoice entries, the fee for each of the one or more selected invoice entries, and the status of the invoice.

11. A system for invoice management related to wearable medical devices, the system comprising:
at least one processor; and
a memory operatively associated with the at least one processor, the memory including instructions stored thereon that, when executed by the at least one processor, cause the at least one processor to:
receive, via an invoice creation interface, a user creation input to create an invoice for one or more tasks associated with a wearable medical device, the user creation input comprising an invoice type;
dynamically display, via the invoice creation interface, a plurality of invoice entry options based on the invoice type, wherein the plurality of invoice entry options are associated with the one or more tasks and/or one or more expenses;
receive, via the invoice creation interface, one or more selected invoice entries of the plurality of invoice entry options;
determine, via a fee schedule, a fee for each of the one or more selected invoice entries; and
generate the invoice comprising the one or more selected invoice entries and the fee associated with the one or more selected invoice entries.

12. The system of claim 11, wherein the at least one processor is further configured to:
subsequent to receiving the one or more selected invoice entries of the plurality of invoice entry options, dynamically display, via the invoice creation interface, a plurality of subfields based on each of the one or more selected invoice entries; and
receive, via the invoice creation interface, one or more user subfield inputs into the plurality of subfields, thereby providing information related to each of the one or more selected invoice entries.

13. The system of claim 12, wherein the at least one processor is further configured to:
add and/or remove, via an administrator interface, invoice entries options of the plurality of invoice entry options; and/or
add and/or remove one or more subfields for at least one invoice entry option of the plurality of invoice entry options.

14. The system of claim 12, wherein the information includes a description of the one or more tasks and/or information related to the one or more expenses.

15. The system of claim 11, wherein the at least one processor is further configured to receive a work order associated with the invoice.

16. The system of claim 15, wherein the at least one processor is further configured to automatically add one or more invoice entries to the invoice based on the work order and/or automatically populate one or more invoice entries based on the work order.

17. The system of claim 15, the at least one processor further configured to:
store at least one of the work order or one or more additional supporting documents in a database;
prior to generating the invoice, detect, via the invoice management system, an inconsistency in the one or more selected invoice entries based on at least one of the work order or the one or more additional supporting documents; and
transmit, via the invoice creation interface, one or more requests to update the one or more selected invoice entries based on the inconsistency.

18. The system of claim 11, wherein the at least one processor is further configured to receive, via an invoice approval interface, an approval of the invoice.

19. The system of claim 18, wherein the at least one processor is further configured to transmit a status communication to a user when a status of the invoice has changed, wherein the status comprises submitted, approved, or rejected.

20. The system of claim 19, wherein the at least one processor is further configured to, upon approval of the invoice, generate a form including the one or more selected invoice entries, the fee for each of the one or more selected invoice entries, and the status of the invoice.
